(19) United States
(12) Patent Application Publication
(10) Pub. No.: US 2004/0058888 A1
(43) Pub. Date: Mar. 25, 2004

(54) Methods for synthesis of alpha-d-gal (1~>3) gal-containing oligosaccharides

(76) Inventors: Laurent Bornaghi, Forest Lake (AU); Gyula Dekany, Westlake (AU); Nicholas Barry Drinnan, Spring Hill (AU); John Papageorgiou, Indooroopilly (AU); Michael Leo West, Hemmant (AU)

Correspondence Address: HELLER EHRMAN WHITE & MCAULIFFE LLP, 275 MIDDLEFIELD ROAD, MENLO PARK, CA, 94025-3506, US

(21) Appl. No.: 10/181,027

(22) PCT Filed: Jan. 12, 2001
(86) PCT No.: PCT/AU01/00028

(30) Foreign Application Priority Data
Jan. 13, 2000 (AU) PQ 5073
Aug. 29, 2000 (AU) PQ 9734

(51) Int. Cl. A61K 31/7008; A61K 31/715; C08B 37/00

(52) U.S. Cl. 514/54; 536/18.7; 536/53

(57) ABSTRACT

This invention relates to reagents and methods for synthesis of biologically active di- and tri-saccharides comprising α-D-Gal(1→3)-D-Gal. In particular the invention provides novel reagents, intermediates and processes for the solution or solid phase synthesis of α-D-galactopyranosyl-(1→3)-D-galactose, and derivatives thereof. In one preferred embodiments the invention provides a protected monosaccharide building block of general formula (II): in which R³ is methoxy or methyl; R¹ is H, benzoyl, pivaloyl, 4-chlorobenzoyl, acetyl, chloroacetyl, levulinoyl, 4-methylbenzoyl, benzyl, 3,4-methylenedioxybenzyl, 4-methoxybenzyl, 4-chlorobenzyl, 4-acetamidobenzyl, or 4-azidobenzyl; and R² is H, Fmoc, benzoyl, pivaloyl, 4-chlorobenzoyl, acetyl, chloroacetyl, levulinoyl, 4-methylbenzoyl, benzyl, 3,4-methylenedioxybenzyl, 4-methoxybenzyl, 4-chlorobenzyl, 4-acetamidobenzyl, or 4-azidobenzyl.

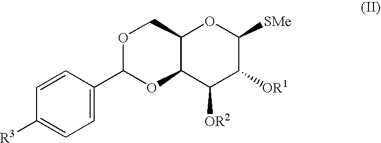

### FIELD OF THE INVENTION

This invention relates to methods for synthesis of biologically active di- and tri-saccharides comprising α-D-Gal(1→3)-D-Gal. In particular the invention provides novel reagents, intermediates and processes for the solution or solid phase synthesis of α-D-galactopyranosyl-(1→3)-D-galactose, and derivatives thereof.

### BACKGROUND OF THE INVENTION

The advent of methods for successful organ transplantation has led to an increasing shortage of donor organs suitable for clinical application. Immuno-concordant species such as non-human primates are potentially a source of allografts which would provide the lowest immunological barrier, but limited availability and ethical concerns, as well as the risk presented by primate retroviruses, mean that this source does not provide a long term solution. Xenografts from discordant but more readily available species, such as pigs, are usually rejected almost immediately. This phenomenon is known as hyperacute rejection (HAR). Thus the suppression of xenoreactive natural antibodies is a key procedure in the implementation of successful xenotransplantation (Tong, Z. et al, 1998). It has been reported that ligands comprising the non-reducing terminal oligosaccharides Galα(1-43)Gal and Galα(1→3)Galβ(1→4)GlcNAc showed the highest affinity with human anti-porcine antibodies (Good, H. et al. 1992). Of the various means proposed for overcoming HAR, the simplest in concept are the competitive blocking of Galα(1→3)Gal antibodies in vivo, or the extracorporeal removal of these antibodies from the circulation (Simon, P. M., 1996). Both methods require the ready availability of the disaccharide or trisaccharide.

In addition to this problem, intestinal infection by Clostridium difficile is one of the most common causes of diarrhoea in hospital patients, especially in the elderly (Boriello, S. P., 1990). C. difficile has been found to be an aetiological agent of antibiotic-associated diarrhoea and pseudomembranous colitis (Smith, J. A. et al., 1997). C. difficile produces two toxins, toxin A and toxin B. Of these, toxin A was shown in animal studies to be an enterotoxin that elicits increased intestinal permeability, fluid secretion and inflammation, and causes severe disruption of the intestinal epithelium (Burakoff, R. et al, 1995; Castex, F. et al, 1994; Eglow, R. et al., 1992; Torres, J. et al, 1990). In model animal systems, the carbohydrate moiety to which toxin A binds has been shown to terminate in the trisaccharide sequence Galα(1→3)Galβ(1→4)GlcNAc (Krivan, H. C. et al, 1986).

Although the chemistry and biochemistry of oligosaccharide compounds has been extensively studied, there are still difficulties associated with their synthesis and purification. Consequently there is a need in, the art for improved methods of synthesis and purification of these compounds.

Apart from the design of effective building blocks, one of the most difficult steps in the synthesis of Galα(1→43)Gal, Galα(1→3)Galβ(1→4)GlcNAc and related compounds is the formation of the α(1→3) linkage. Although a number of synthetic routes have been described, all of these methods are complex, time-consuming, and costly, and are unsuited to large-scale synthesis.

Chacon-Fuertes provided a procedure for the synthesis of 3-O-α-D-galactopyranosyl-D-galactose [i]

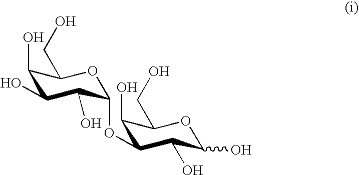

which required a mercuric cyanide-catalysed glycosylation for formation of the α(143) glycosidic linkage (Chacon-Fuertes M. E. and Martin-Lomas, M., 1975). The synthesis was protracted, required chromatography, and used dangerous reagents. Lemieux described the chemical synthesis of 3-O-α-D-galactopyranosyl-D-galactose using a per-O-benzylated α-D-galactopyranosyl bromide sugar donor and a 2,2,2-trichloroethyl 2,4,6-tri-O-acetyl-β-D-galactopyranoside acceptor (Lemieux, R. U. and Driguez, H., 1975). Lemieux employed tetraethyl ammonium bromide as a promoter in a reaction that after chromatography gave 35% yield of product. ¹H NMR spectroscopy indicated that the glycosylation product still contained substantial impurities. After deprotection with zinc/acetic acid and preparative thin layer chromatography, de-O-acetylation, hydrogenolysis and paper chromatography, an authentic sample of 3-O-α-D-galactopyranosyl-D-galactose was finally achieved.

An alternative approach used an allyl 2-O-benzoyl-4,6-O-benzylidene-β-D-galactopyranoside acceptor and an acetimidate sugar donor (Sinay, P. and Jacquinet, J. C., 1979). The formation of the α(1→43) linkage was effected with toluene sulphonic acid in nitromethane in good yield, but chromatography was required for purification. Although generally maintaining yields of greater than 90% for the remainder of the synthesis to the target 3-O-α-D-galactopyranosyl-D-galactose, chromatography was required at most steps. Similarly a benzylated Gal(α1-3)Gal disaccharide was synthesised using an α-D-galactopyranosyl bromide donor, but employing stannylene chemistry to selectively activate the 3-O-position of the acceptor galactoside, (Augé, C. and Veyrières, A., J. C. S., 1979). The benzylated Galα(1→3)Gal disaccharide subsequently underwent hydrogenolysis to afford 3-O-α-D-galactopyranosyl-D-galactose. The reported yields were very low, and most steps required chromatography.

Another synthesis of the 3-O-α-D-galactosyl-D-galactose disaccharide employed a benzyl 2,4,6-tri-O-benzyl-β-D-galactopyranoside acceptor and a fully-benzylated imidate galactosyl donor (Milat, M-L. et al, 1982). The free disaccharide was eventually obtained after a final hydrogenolysis, and although reasonable yields were achieved, chromatography was unavoidable at many stages of the synthesis. Takeo employed a galactosyl bromide donor and tetraethylammonium bromide as a promoter, and synthesised the disaccharide of interest in a protected form in 40% yield after chromatography. Hydrogenolysis then yielded 3-O-α-D-galactopyranosyl-D-galactose (Takeo, K. and Maeda, H., 1988). A chemo-enzymatic synthesis utilised α-D-galactosidase from coffee beans to form the disaccharide, in unreported yield. p-Nitrophenyl-α-D-galactopyranoside was used as both the acceptor and donor. The resultant disaccharide derivative was then modified and chromatographed to afford 3-O-α-D-galactopyranosyl-D-galactose (Matsuo, I. et al, 1997).

It is desirable to avoid the use of toxic reagents, and in order to reduce costs it is also highly desirable to minimise the number of purification steps. If possible, it is particularly desirable to minimize the number of chromatographic purification steps, or even to avoid entirely the need for chromatographic purification, because this technique is time-consuming and costly.

Synthesis of the trisaccharide α-D-galactopyranosyl-(1→3)-β-D-galactopyranosyl-(1→4)-N-acetyl-D-glucosamine (ii) has understandably been even more difficult than that of α-D-galactopyranosyl-(1→3)-D-galactose.

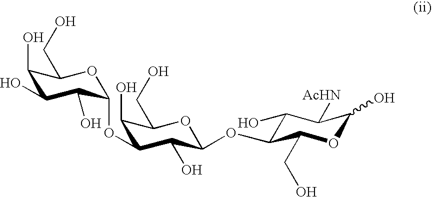

There have been no methods reported in the literature for the synthesis of (ii) using chemical means, although closely analogous compounds have been developed for in vitro and in vivo applications (Garegg, P. J. and Oscarson, S., 1985; Schaubach, R. et al, 1991). There have been some reports of enzymatic synthesis of oligosaccharide (ii) and derivatives thereof. Nilsson synthesised the 2-N-trichloroethoxycarbonyl protected ethyl thioglycoside of (ii) by enzymatic methods, using an α-D-galactosidase to effect the formation of the α(1→43) glycosidic linkage followed by β-D-galactosidase treatment (Nilsson, K. G. I., 1997). Similarly galactosidases have been used for the synthesis of target compound (ii), employing similar methodologies (Matsuo, I. et al, 1997). Another ethyl thioglycoside derivative of (ii) was synthesised using a and β galactosidases (Vic, G. et al, 1997). Analogues of (ii) similar to those described above with lipophilic tails attached via the glycosidic linkage were synthesised using α(1→3) galactosyltransferases (Sujino, K. et al., 1998).

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

We have now found that novel thioacyl-substituted glycosides of 3-O-α-D-galactopyranosyl-D-galactose can be used for glycoconjugate synthesis by chemical methods. These derivatives can be linked to a suitable soluble support, such as polyethylene glycol. These compounds can be used for removal of anti-Gal antibodies from a transplant recipient's blood prior to xenotransplantation, or as anti-bacterial agents to combat bacteria such as C. difficile.

### SUMMARY OF THE INVENTION

In a first aspect the invention provides a protected glucosamine compound of general formula I:

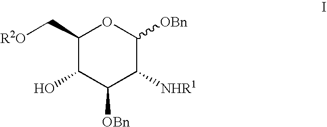

in which R¹ is H or acetyl and R² is benzyl or 4-chlorobenzoyl,

with the proviso that when R² is benzyl, R¹ is not acetyl.

In a second aspect, the invention provides a protected monosaccharide building block of general formula II:

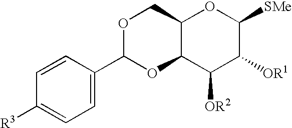

in which R³ is H, methoxy or methyl, and in which

(a) when R³ is methoxy or methyl, R¹ is H, benzoyl, pivaloyl, 4-chlorobenzoyl, acetyl, chloroacetyl, levulinoyl, 4-methylbenzoyl, benzyl, 3,4-II methylenedioxybenzyl, 4-methoxybenzyl, 4-chlorobenzyl, 4-acetamidobenzyl, or 4-azidobenzyl; and

R² is H, Fmoc, benzoyl, pivaloyl, 4-chlorobenzoyl, acetyl, chloroacetyl, levulinoyl, 4-methylbenzoyl, benzyl, 3,4-methylenedioxybenzyl, 4-methoxybenzyl, 4-chlorobenzyl, 4-acetamidobenzyl, or 4-azidobenzyl;

(b) when R³ is H, R¹ is benzoyl, pivaloyl, 4-chlorobenzoyl, acetyl, chloroacetyl, levulinoyl, benzyl, 3,4-methylene-dioxybenzyl, 4-methoxybenzyl, 4-chlorobenzyl, 4-acetamidobenzyl, or 4-azidobenzyl, and

R² is Fmoc, benzoyl, 4-chlorobenzoyl, acetyl, chloroacetyl, levulinoyl, 4-methylbenzoyl, benzyl, 3,4-methylenedioxybenzyl, 4-methoxybenzyl, 4-chlorobenzyl, 4-acetamidobenzyl, or 4-azidobenzyl,

with the provisos that

(i) when R¹ is acetyl, R² is not chloroacetyl or acetyl, and vice versa;

(ii) when R² is levulinoyl, R¹ is not benzoyl, and vice versa; and

(iii) when R¹ is benzoyl, R² is not benzoyl, and vice versa.

When R² is Fmoc, R¹ is benzoyl, pivaloyl, 4-chlorobenzoyl, acetyl, chloroacetyl, levulinoyl, 4-methylbenzoyl, benzyl, 3,4-methylene-dioxybenzyl, 4-methoxybenzyl, 4-chlorobenzyl, 4-acetamidobenzyl, or 4-azidobenzyl.

Preferably the compound is of general formula III:

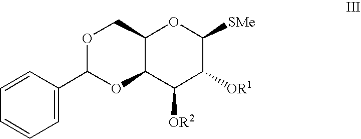

in which R¹ is pivaloyl, benzoyl, 4-chlorobenzoyl, 4-methoxybenzyl, or 3,4-methylenedioxybenzyl, and

R² is H, Fmoc, 4-chlorobenzoyl, acetyl, chloroacetyl, levulinoyl, 4-methoxybenzyl, or 3,4-methylenedioxybenzyl, with the proviso that if R¹ is benzoyl, R² is not levulinoyl.

In preferred embodiments, the compound is

(a) a galactopyranoside of general formula III, in which R¹ is 4-chlorobenzoyl, pivaloyl or acetyl, and R² is Fmoc or H;

(b) a compound of general formula III in which R¹ is 4-chlorobenzoyl and R² is chloroacetyl; or

(c) a compound of general formula III in which both R¹ and R² are 3,4-methylenedioxybenzyl.

In a third aspect, the invention provides a galactopyranoside compound of general formula IV:

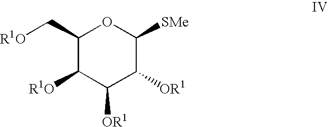

in which each R¹ is independently 4-chlorobenzyl, 4-azidobenzyl, 4-N-acetamidobenzyl, 4-methylbenzyl, 3,4-methylenedimethoxybenzyl, or 2-nitrobenzyl.

Preferably each R¹ is 4-chlorobenzyl.

In a fourth aspect the invention provides a polyethyleneglycol (PEG)-linked monosaccharide of general formula V:

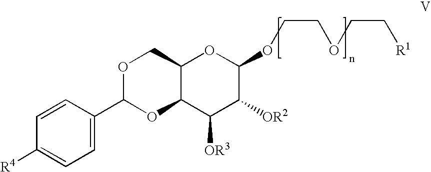

in which n is an integer from 1-5;

R¹ is a linking group or a group suitable for the formation of a covalent linkage, and includes but is not limited to groups such as halogen, azido, carboxylic acid, thiol, hydroxyl, thioester, xanthate, amido, or dithiocarbamate; R² is acetyl, 4-chlorobenzoyl, levulinoyl, pivaloyl, chloroacetate, benzoyl, or 4-methybenzoyl;

R³ is H, Fmoc, benzoyl, pivaloyl, 4-chlorobenzoyl, acetyl, chloroacetyl, levulinoyl, 4-methylbenzoyl, 3,4-methylenedioxybenzyl, 4-methoxybenzyl, 4-acetamidobenzyl, or 4-azidobenzyl; and

R⁴ is methoxy, H, or methyl.

Preferably n is 2, R¹ is thiobenzoate or thiobiphenylcarbonyl, R² is 4-chlorobenzoyl, R³ is H, and R⁴ is H.

In a fifth aspect the invention provides a compound of general formula VI:

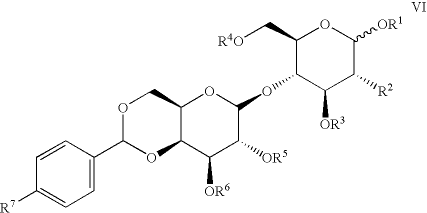

in which R⁷ is H, methoxy or methyl;

R¹ is aryl, substituted aryl, benzyl, substituted benzyl, alkyl, substituted alkyl, PEG, or substituted PEG;

R² is acetamido or amino;

R³ and R⁴ are independently benzyl, substituted benzyl, silylether or acyl;

R⁵ is 4-chlorobenzoyl, benzoyl, pivaloyl, acetyl, levulinoyl or 4-methylbenzoyl; and

R⁶ is a substituted or unsubstituted pyranosyl or furanosyl sugar, H, Fmoc, acetyl, chloroacetyl, levulinoyl, 3,4-methylenedioxybenzyl, 4-methoxybenzyl, 4-acetamidobenzyl, or 4-azidobenzyl.

When the anomeric configuration of the glucosamine moiety of general formula VI is α and R³ is benzyl and R⁴ is benzoyl and R⁷ is H, then R² may be acetamido, amino, N-phthalimido, R⁵ may be 4-chlorobenzoyl, benzoyl, pivaloyl, acetyl, levulinoyl or 4-methylbenzoyl, and R⁶ is a substituted or unsubstituted pyranosyl or furanosyl sugar, H, Fmoc, acetyl, chloroacetyl, levulinoyl, 3,4-methylenedioxybenzyl, 4-methoxybenzyl, 4-acetamidobenzyl, or 4-azidobenzyl.

When the anomeric configuration of the glucosamine moiety of general formula VI is β and R¹ is benzyl and R⁷ is H, then R² is acetamido, amino, or N-phthalimido; R³ and R⁴ are independently benzyl, substituted benzyl, silylether or acyl; R⁵ is 4-chlorobenzoyl, benzoyl, pivaloyl, acetyl, levulinoyl or 4-methylbenzoyl, and R⁶ is a substituted or unsubstituted pyranosyl or furanosyl sugar, H, Fmoc, acetyl, chloroacetyl, levulinoyl, 3,4-methylenedioxybenzyl, 4-methoxybenzyl, 4-acetamidobenzyl, or 4-azidobenzyl.

When the anomeric configuration of the glucosamine moiety of general formula VI is α and R¹, R³, and R⁴ are benzyl or substituted benzyl and R⁷ is H, then R² is acetamido, amino, or N-phthalimido, R⁵ is pivaloyl, 4-chlorobenzoyl, benzoyl, or levulinoyl, and R⁶ is a substituted or unsubstituted pyranosyl or furanosyl sugar, H, Fmoc, acetyl, chloroacetyl, levulinoyl, 3,4-methylenedioxybenzyl, 4-methoxybenzyl, 4-acetamidobenzyl, or 4-azidobenzyl, with the proviso that when R³ and R⁴ are benzyl, R⁵ is not acetyl or benzoyl.

In preferred embodiments:

(a) the anomeric configuration of the glucosamine moiety of general formula VI is β, R¹ is benzyl, R² is amino or acetamido, R³ and R⁴ are benzyl, R⁵ is 4-chlorobenzoyl, pivaloyl or acetyl, R⁶ is Fmoc or H, and R⁷ is H;

(b) the anomeric configuration of the glucosamine moiety of general formula VI is α, R¹ is benzyl, R² is acetamido, R³ is benzyl, R⁴ is benzoyl or benzyl, R⁵ is 4-chlorobenzoyl, R⁶ is H or 4-chloroacetyl and R⁷ is H;

(c) the compound is a trisaccharide of General Formula VII:

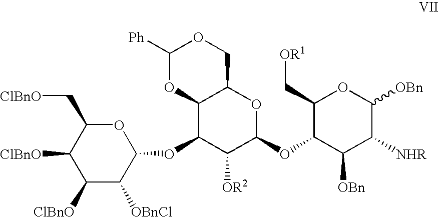

in which R is H or acetyl; R¹ is hydrogen, benzyl, benzoyl or p-chlorobenzoyl; and R² is hydrogen, 4-chloro-benzoyl, acetyl, benzoyl or pivaloyl;

(d) the compound is a trisaccharide of general formula VII, in which the anomeric configuration of the reducing end is a, R is acetyl, R¹ is benzoyl, 4-chlorobenzoyl or H, and R² is 4-chlorobenzoyl or H; or

(e) the compound is a trisaccharide of general formula VII, in which the anomeric configuration of the reducing sugar is β, R is acetyl or H, R¹ is benzyl, and R² is H, 4-chlorobenzoyl, pivaloyl or acetyl.

In a sixth aspect the invention provides a compound of general formula VIII:

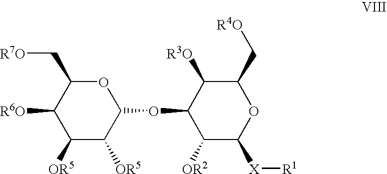

in which R⁵, R⁶ and R⁷ are independently H, 4-chlorobenzyl, 4-methoxybenzyl, 4-methylbenzyl, 4-acetamidobenzyl, azidobenzyl or 3,4-methylenedioxybenzyl;

X is O, S, or N;

R¹ is alkyl, substituted alkyl, aryl, substituted aryl, PEG or substituted PEG;

R² is levulinoyl, 4-chlorobenzoyl, benzoyl, 4-methylbenzoyl, acetyl or pivaloyl; and

R³ and R⁴ may combine to form a benzylidene ring, which may optionally be substituted at the 4 position by methyl or methoxy; alternatively R³ and R⁴ may independently be H, benzyl or substituted benzyl.

When R⁵ is 4-chlorobenzyl, 4-methoxybenzyl, 4-methylbenzyl, 4-acetamidobenzyl, azidobenzyl or 3,4-methylenedioxybenzyl, and R⁶ and R⁷ combine to form a benzylidene or substituted benzylidene ring, then X is O, S, or N, R¹ is alkyl, substituted alkyl, aryl, substituted aryl, PEG, substituted PEG, acyl or substituted acyl, and R² is levulinoyl, 4-chlorobenzoyl, benzoyl, 4-methylbenzoyl, acetyl or pivaloyl.

When X is oxygen and R¹ is 3,4-methylenedioxybenzyl, then R² is H, 4-chlorobenzoyl, pivaloyl, acetyl, levulinoyl, benzoyl or chloroacetyl, R³ and R⁴ may combine to become a benzylidene ring or may independently be H, benzyl or substituted benzyl, and R⁵, R⁶ and R⁷ may be H, benzyl, 4-chlorobenzyl, 4-methoxybenzyl, 4-acetamidobenzyl, azidobenzyl or 3,4-methylenedioxybenzyl.

When X is oxygen and R¹ is 2-[2-(2-thiobenzoyl)-ethoxy)ethyl or 2-[2-(2-thiobiphenylcabonyl)ethoxy], then R² is H, 4-chlorobenzoyl, pivaloyl, acetyl, levulinoyl, benzoyl or chloroacetyl, R³ and R⁴ may combine to form a benzylidene ring or may independently be H, benzyl, 4-chlorobenzyl, 4-methoxybenzyl, 4-acetamidobenzyl, azidobenzyl or 3,4-methylenedioxybenzyl, R⁵ is H, benzyl, 4-chlorobenzyl, 4-methoxybenzyl, 4-acetamidobenzyl, azidobenzyl or 3,4-methylenedioxybenzyl, and R⁶ and R⁷ may combine to become a benzylidene ring or may independently be H, benzyl, 4-chlorobenzyl, 4-methoxybenzyl, 4-acetamidobenzyl, azidobenzyl or 3,4-methylenedioxybenzyl.

When X is sulphur, R¹ is alkyl, substituted alkyl, aryl or substituted aryl, R³ and R⁴ combine to form a benzylidene ring and R⁵, R⁶ and R⁷ are benzyl, then R² is levulinoyl, 4-chlorobenzoyl, benzoyl, acetyl or pivaloyl, with the proviso that when R¹ is phenyl, R² is not levulinoyl.

Preferably either

(a) X is oxygen, R¹ is 2-[2-(2-thiobenzoyl)ethoxy)ethyl or 2-[2-(2-thiobiphenylcabonyl)ethoxy], R² is H or 4-chlorobenzoyl, R³ and R⁴ are H or combine to form a benzylidene ring, R⁵ is H or 3,4-methylenedioxybenzyl, and R⁶ and R⁷ are both H or combine to form a benzylidene ring;

(b) X is S, R¹ is methyl, R² is 4-chlorobenzoyl, R³ and R⁴ combine to form a benzylidene ring, and R⁵, RE and R⁷ are each 4-chlorobenzyl; or

(c) X is oxygen, R¹ is 3,4-methylenedioxybenzyl, R² is 4-chlorobenzoyl or H, R³ and R⁴ combine to form a benzylidene ring or are both H, and R⁵, RE and R⁷ are independently 4-chlorobenzyl or H.

In a seventh aspect the invention provides a compound of general formula IX:

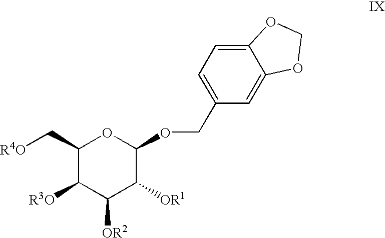

in which R¹ is 4-chlorobenzoyl, pivaloyl, acetyl, levulinoyl, benzoyl or chloroacetyl;

R² is H, benzyl, 4-chlorobenzyl, 4-methoxybenzyl, 4-acetamidobenzyl, azidobenzyl, 3,4-methylenedioxybenzyl, Fmoc, levulinoyl, acetyl or chloroacetyl; and

R³ and R⁴ may combine to form a benzylidene ring, or may independently be H, benzyl, 4-chlorobenzyl, 4-methoxybenzyl, 4-acetamidobenzyl, azidobenzyl or 3,4-methylenedioxybenzyl.

Preferably R¹ is 4-chlorobenzoyl, R² is H, and R³ and R⁴ combine to form a benzylidene ring.

In an eighth aspect the invention provides a polyethyleneglycol(PEG)-linked disaccharide of General Formula X or a trisaccharide of General Formula XI:

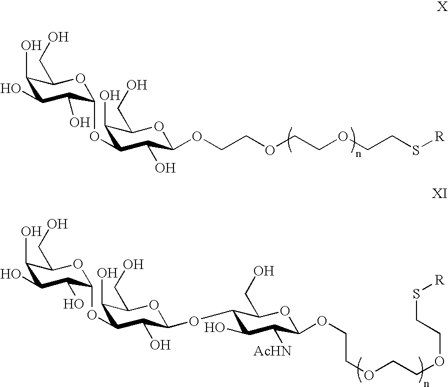

in which R is hydrogen or acyl, and n is an integer of from 1 to 3.

Preferably the compound of General Formula [symbol] 2-[2-(2-thiobiphenylcarbonyl)ethoxy]-ethyl 3-O-(α-D-galactopyranosyl)-α-galactopyo [symbol] de.

In a ninth aspect, the XI [symbol] ion provides Galα(1→3)Galβ(1→44)GlcNAc coupled to a solid support to give a compound of general formula XII:

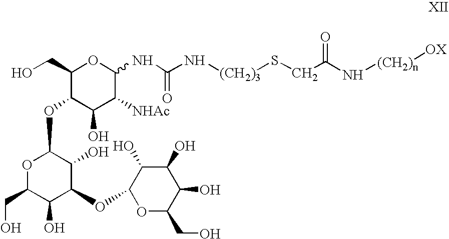

in which X is a solid support such as Sepharose or silica gel, and n is an integer of from 3 to 6.

The compounds of the first seven aspects of the invention are useful as intermediates in the synthesis of di- and trisaccharides. Accordingly, in a tenth aspect, the invention provides a method of synthesis of a desired compound of General Formula X to General Formula XII, or of α-D-galactopyranosyl-(1→3)-β-D-galactopyranosyl-(1→4)-N-acetyl-D-glucosamine (Galα(1→43)Galβ(1→4)GlcNAc), α-D-galactopyranosyl-(1→3)-β-D-galactopyranose (Galα(1→43)Gal), or β-D-galactopyranosyl-(1→4)—N-acetyl-D-glucosamine (Galβ(1→4)GlcNAc), comprising the step of using a compound of General Formula I to IX as an intermediate.

Preferably when the desired compound is of general Formula X or XI the intermediate compound is of General Formula V. It will be clearly understood that although a compound of General Formula VI may be synthesised using a compound of General Formula I as an intermediate, alternative syntheses are available.

For the purposes of this specification, the term “alkyl” is intended to include saturated, unsaturated and cyclic hydrocarbon groups, and combinations of such groups. Suitable substituents on hydrocarbon chains or aryl rings include Br, Cl, F, I, CF₃, NH₂, substituted amino groups such as NHacyl, hydroxy, carboxy, C₁₋₆alkylamino and C₁₋₆alkoxy groups such as methoxy, and are preferably F, Cl, hydroxy, C₁₋₆alkoxy, amino, C₁₋₆alkylamino or C₁₋₆carboxy.

In a eleventh aspect, the invention provides a method of preventing or reducing a hyperacute rejection response associated with xenotransplantation, comprising the step of administering an effective dose of thioalkyl Galα-(1→3)Gal or thioalkyl Galα(1→3)Galβ(1→4)GlcNAc to a subject in need of such treatment.

The compound may be administered before, during or after xenotransplantation.

In a twelfth aspect, the invention provides a method of preventing or reducing hyperacute rejection associated with xenotransplantation, comprising the steps of

a) removing plasma from a patient who is to undergo xenotransplantation;

b) exposing the plasma to thioalkyl Galα(1→3)Gal or thioalkyl-Galα(1→3)Galβ(1→4)GlcNAc linked to a solid support, and

c) reinfusing the thus-treated plasma into the patient.

In a thirteenth aspect, the invention provides a method of depleting anti-Galα(1→3)Gal antibodies from a plasma or serum sample, comprising the step of exposing the plasma or serum to thioalkyl Galα(1→3)Gal or thioalkyl Galα(1→3)Galβ(1→4)GlcNAc linked to a soluble support.

In a fourteenth aspect, the invention provides a method of treatment of C. difficile infection, comprising the step of administering an effective amount of α-D-galactopyranosyl-(1→3)-β-D-galacto-pyranosyl-(1→4)-N-acetyl-D-glucosamine (Galα(1→3)Galβ(1→4)GlcNAc) or of thioalkyl Galα(1→3)Galβ(1→4)GlcNAc, preferably linked to a soluble support, to a subject in need of such treatment.

Preferably the soluble support is a multidentate ligand or a dendrimer compound. Suitable dendrimers are disclosed for example in International patent application No. PCT/AU95/00350 (WO95/34595) by Biomolecular Research Institute Ltd.

In the eleventh to the fourteenth aspects of the invention, the subject may be a human, or may be a domestic, companion or zoo animal. While it is particularly contemplated that the compounds of the invention are suitable for use in medical treatment of humans, they are also applicable to veterinary treatment, including treatment of companion animals such as dogs and cats, and domestic animals such as horses, cattle and sheep, or zoo animals such as felids, canids, bovids, and ungulates.

Methods and pharmaceutical carriers for preparation of pharmaceutical compositions are well-known in the art, as set out in textbooks such as Remington's Pharmaceutical Sciences, 19th Edition, Mack Publishing Company, Easton, Pa., USA.

The compounds and compositions of the invention may be administered by any suitable route, and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered.

The carrier or diluent, and other excipients, will depend on the route of administration, and again the person skilled in the art will readily be able to determine the most suitable formulation for each particular case.

For the purposes of this specification it will be clearly understood that the word “comprising” means “including but not limited to”, and that the word “comprises” has a corresponding meaning.

### DETAILED DESCRIPTION OF THE INVENTION

The invention will now be described in detail by way of reference only to the following non-limiting examples. Abbreviations used herein are as follows:

| | |
|---|---|
| AcN | Acetonitrile |
| Bn | Benzyl |
| CH₂Cl₂ | Dichloromethane |
| CHCl₃ | Chloroform |
| pClBn | para-chlorobenzyl |
| pClBz | para-chlorobenzoyl |
| DCM | Dichloromethane |
| DMF | N,N′-Dimethylformamide |
| DMTST | Dimethyl (methylthio) sulphoniumtrifluoro- |
| | methanesulphonate |
| EtOAc | Ethyl acetate |
| EtOH | Ethanol |
| H₂O | Water |
| HRMS | High resolution mass spectrometry |
| MDBn | 3,4-methylenedioxybenzyl |
| Me | Methyl |
| MeCN | Acetonitrile |
| MeOH | Methanol |
| MgSO₄ | Magnesium sulphate |
| NaHCO₃ | Sodium hydrogen carbonate |
| NMR | Nuclear magnetic resonance |
| PEG | Polyethylene glycol |
| Ph | Phenyl |
| SOCl₂ | Thionyl chloride |
| TBDMS | tertiary-butyldimethylsilyl |
| THF | Tetrahydrofuran |

### EXAMPLE 1

### Preparation of 3,4-Methylenedioxybenzyl 4,6-O-Benzylidene 2-O-(4-chlorobenzoyl)-β-D-Galactopyranoside Acceptor

The strategy for this preparation is set out in Reaction Scheme 1.

Synthesis of α-D-Galactopyranosyl-(1→3)-D-Galactose

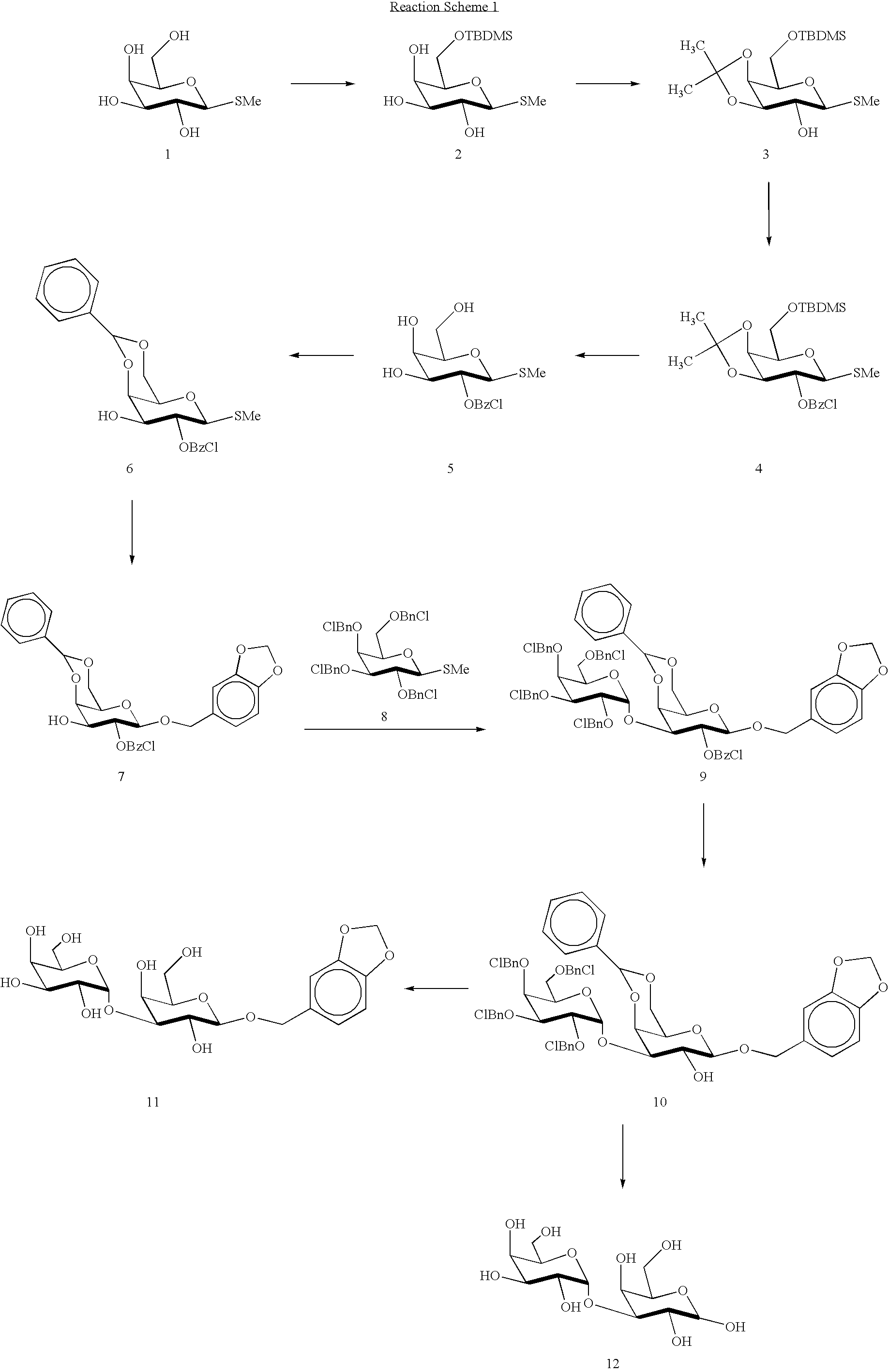

Methyl 6-O-tert-butyldimethylsilyl-1-thio-β-D-galactopyranoside (2)

A mixture of t-butyldimethylsilyl chloride (68.35 g, 453.51 mmol) and 4-dimethylaminopyridine (55.40 g, 453.51 mmol) in dry 1,2-dichloroethane (800 ml) was stirred at 80° C. for 15 minutes. Methyl 1-thio-β-D-galactopyranoside (1) (100 g, 476.19 mmol) was added in 5 portions in 15 minutes to the stirred solution at 80° C., and the reaction mixture was stirred under reflux for 1 hour. The resulting clear solution was cooled to room temperature, diluted with CHCl₃ (2 000 ml), washed four times with diluted brine solution (water-brine 2:1) (750 ml). The aqueous layers of the last two washings were collected and extracted with CHCl₃ (400 ml). The organic layers were combined, dried over MgSO₄ and evaporated. The residue was kept under high vacuum for 15 min, then was dissolved in dry MeCN (200 ml). The solution was evaporated, and the residue was kept under high vacuum for 15 min. This drying process was repeated using another 200 ml of dry MeCN, to give the crude methyl 6-O-tert-butyldimethylsilyl-1-thio-β-D-galactopyranoside (2) (117.5 g, 80%) as a syrup.

R_{f} 0.65 (MeCN/H₂O 10:1) MS (electrospray) C₁₃H₂₈O₅SSi (324.23) m/z (%) 347[M+Na]⁺ (100), 325[M+H]⁺ (75).

Methyl 6-O-tert-butyldimethylsilyl-3,4-O-isopropylidene-1-thio-β-D-galactopyranoside (3)

A mixture of crude methyl 6-O-tert-butyldimethylsilyl-1-thio-β-D-galactopyranoside (2) (117.46 g, 362.27 mmol), 2,2-dimethoxypropane (66.82 ml, 543.41 mmol) and p-toluenesulphonic acid (200 mg) in dry MeCN (800 ml) was stirred at 40° C. for 30 minutes. The reaction mixture was neutralized with triethylamine (3 ml) and evaporated to give a white crystalline residue (3) (161.3 g)

R_{f} 0.62 (EtOAc/Hexane 2:1) MS (electrospray) C₁₆H₃₂O₅SSi (364.58) m/z (%) 387[M+Na]⁺ (45), 365 M+H]⁺ (100).

Methyl 6-O-tert-butyldimethylsilyl-2-O-(4-chlorobenzoyl)-3,4-O-isopropylidene-1-thio-β-D-galactopyranoside (4)

A mixture of methyl 6-O-tert-butyldimethylsilyl-3,4-O-isopropylidene-1-thio-β-D-galactopyranoside (3) (155.44 g, 427.03 mmol) and 4-dimethylaminopyridine (62.60 g, 512.44 mmol) in dry 1,2-dichloroethane (750 ml) was stirred at room temperature. 4-Chlorobenzoyl chloride (89.68 g, 512.44 mmol) was added to the stirred reaction mixture in 15 minutes. After the addition the resulting suspension was stirred under reflux for 30 minutes. The reaction mixture was cooled to 10° C. and filtered. The crystalline solid was washed on the funnel with dry 1,2-dichloroethane (300 ml) and filtered. The filtrates were combined, diluted with CHCl₃ (2000 ml) and washed twice with-diluted brine solution (water-brine 2:1) (1500 ml). The organic layer was dried over MgSO₄ and evaporated. The residue was kept under high vacuum for 15 minutes. The resulting syrup was dissolved in dry MeCN (200 ml) and evaporated using high vacuum at the end of the evaporation, to give the crude methyl 6-O-tert-butyldimethylsilyl-2-O-(4-chlorobenzoyl)-3,4-O-isopropylidene-1-thio-β-D-galactopyranoside (4) (165 g) as a colourless syrup.

R_{f} 0.68 (Hexane/EtOAc 2:1) MS (electrospray) C₂₃H₃₅O₆SSi (50.3.14), m/z (%) 503[M+H]⁺ (100), 525[M+Na]⁺ (38).

Methyl 2-O-(4-chlorobenzoyl)-1-thio-β-D-galactopyranoside (5)

A mixture of methyl 6-O-tert-butyldimethylsilyl-2-O-(4-chlorobenzoyl)-3,4-isopropylidene-β-D-galactopyranoside (4) (173 g, 344.62 mmol) and p-toluenesulphonic acid (600 mg) in MeOH-MeCN 3:1 (2000 ml) was stirred under reflux for 1 hour. The reaction mixture was cooled to room temperature and evaporated. The resulting white solid residue was suspended in diisopropylether (1000 ml) and filtered. The crystalline solid was washed twice with diisopropylether (300 ml), then with diethylether (500 ml) and dried to give methyl 2-O-(4-chlorobenzoyl)-1-thio-β-D-galactopyranoside (5) (107 g) as a white crystalline powder.

R_{f} 0.45 (MeCN/H₂O 10:1) MS (electrospray) C₁₄H₁₇ClO₆S (348.80) m/z (%) 371[M+Na]⁺ (35), 349[M+H]⁺ (100).

Methyl 2-O-(4-chlorobenzoyl)-4,6-O-benzylidene-1-thio-β-D-galactopyranoside (6)

A mixture of methyl 2-O-(4-chlorobenzoyl)-1-thio-β-D-galactopyranoside (5) (94.16 g, 270.57 mmol), α,α-dimethoxytoluene (60.9 ml, 405.86 mmol) and p-toluenesulphonic acid (200 mg) in dry MeCN (500 ml) was stirred at 70° C. for 30 minutes. The reaction mixture was cooled to room temperature, neutralized with triethylamine (3 ml) and evaporated. The residue was taken up in CHCl₃ (1500 ml), washed with diluted brine solution (water-brine. 2:1) (750 ml), with saturated NaHCO₃ solution (500 ml), with diluted brine again (water-brine 2:1) (750 ml), dried over MgSO₄ and evaporated. The resulting white solid was kept under high vacuum for 15 minutes. The dry residue was crystallized from MeCN (250 ml) at room temperature to give 68.5 g pure product. Water (80 ml) was added slowly to the mother liquor, and the solution was left at room temperature to crystallize another 20.8 g of methyl 2-O-(4-chlorobenzoyl)-4,6-O-benzylidene-1-thio-β-D-galactopyranoside (6) (yield: 75%).

R_{f} 0.65 (EtOAc/Hexane 2:1) MS (electrospray) C₂₁H₂₁ClO₆S (436.91) m/z (%) 437[M+H]⁺ (56), 459[M+Na]⁺ (100).

¹H N (CDCl₃) δ 8.01-7.37 (9H, aromatic), 5.56 (s, 1H, benzylidene), 5.44 (t, 1H, H-2), 4.5 (d, 1H, J₁₋₂=9.0, H-1), 4.38 (dd, 1H, H-6a), 4.30 (dd, 1H, H-4), 4.04 (dd, 1H, H-b), 3.90 (m, 1H, H-3), 3.6 (s, 1H, H-5), 2.25 (s, 3H, S—H₃).

3,4-Methylenedioxybenzyl 4,6-O-benzylidene 2-O-(4-chlorobenzoyl)-β-D-galactopyranoside (7)

To a mixture of methyl 4,6-O-benzylidene 2-O-(4-chlorobenzoyl)-1-thio-β-D-galactopyranoside (6) (10 g, 22.9 mmol), 3,4-methylenedioxybenzyl alcohol (5.6 g, 36.8 mmol) and powdered molecular sieves (5 Å, 15 g) in dry 1,2-dichloroethane (200 mL) at 0° C., was added methyl trifluoromethanesulphonate (6 g, 36.6 mmol) in one portion under nitrogen atmosphere. The reaction mixture was sealed and left to warm to room temperature, and stirred for 3 h. The mixture was then neutralized with triethylamine (15 mL), diluted with CHCl₃ (350 mL) and filtered through celite. The filtrate was washed with saturated NaHCO₃ solution (4×500 mL), and the organic layer was dried over MgSO₄ and evaporated to dryness leaving a white solid. The solid was suspended in diisopropylether (200 mL), filtered, washed with diisopropylether (200 mL) and dried to give 3,4-methylenedioxybenzyl 4,6-O-benzylidene 2-O-(4-chlorobenzoyl)-β-D-galactopyranoside (7) (7.5 g, 61% yield) as a white powder.

R_{f} 0.60 (CH₂Cl₂/EtOH 20:1) MS (electrospray) C₂₈H₂₅ClO₉ (540.95) m/z (%) 437[M+H]⁺ (56), 558[M+H+NH₃]⁺ (100).

### EXAMPLE 2

### Preparation of Methyl 2,3,4,6-tetra-O-(4-chlorobenzyl)-1-thio-β-D-Galactopyranoside Glycosyl Donor

Methyl 2,3,4,6-tetra-O-(4-chlorobenzyl)-1-thio-β-D-galactopyranoside (8)

To a stirred suspension of sodium hydride (95%) (14.43 g, 571.42 mol) in dry DMF (300 ml) a solution of methyl 1-thio-β-D-galactopyranoside (1) (20 g, 95.23 mmol) in dry DMF (200 ml) was added dropwise at 0° C. in nitrogen atmosphere. At the end of the addition the ice-bath was removed and the reaction mixture was stirred at room temperature for 30 minutes. 4-Chlorobenzyl chloride (97.74 g, 571.42 mmol) was added dropwise to the stirred reaction mixture keeping the temperature 10-20° C. After the addition, the reaction mixture was stirred at room temperature overnight. The resulting suspension was cooled with ice-bath and methanol (11 ml) was added slowly. When the hydrogen formation had stopped, the suspension was evaporated to dryness at 45-50° C. The remaining DMF was removed by co-evaporation with xylene (100 ml). The residue was taken up in CH₂Cl₂ (500 ml), washed twice with water (500 ml), saturated NaHCO₃ solution (500 ml), dried over MgSO₄ and evaporated. The residue was crystallized from EtOH (500 ml) to give methyl 2,3,4,6-tetra-O-(4-chlorobenzyl)-1-thio-β-D-galactopyranoside (8) (40 g, 60%) as a white crystalline solid.

R_{f} 0.72 (Hexane/EtOAc 3:1) MS (electrospray) C₃₅H₃₄Cl₄O₅S (708.53) m/z (%) 709[M+H]⁺ (100), 731[M+Na]⁺ (48).

### EXAMPLE 3

### Preparation of 3-O-(α-D-galactopyranosyl)-D-galactopyranose

3,4-Methylenedioxybenzyl 4,6-O-benzylidene-2-O-(4-chlorobenzoyl)-3-O-(2,3,4,6-tetra-O-(4-chlorobenzyl)-α-D-galactopyranosyl)-β-D-galac-topyranoside (9)

Methyl trifluoromethanesulphonate (4 g, 24 mmol) was added under nitrogen to a mixture of 3,4-methylenedioxybenzyl 4,6-O-benzylidene 2-O-(4-chlorobenzoyl)-β-D-galactopyranoside (7) (6.5 g, 12 mmol), methyl 2,3,4,6-tetra-o-(4-chlorobenzyl)-thio-p-D-galactopyranoside (8) (12 g, 17 mmol) and powdered molecular sieves (5 Å, 10 g) in dry 1,2-dichloroethane (250 mL). The sealed reaction mixture was left to warm to room temperature and then stirred for 80 minutes. The reaction mixture was neutralized with triethylamine (12 g) and diluted with CHCl₃ (500 mL). The suspension was filtered through celite and the filtrate was washed with saturated NaHCO₃ solution (3×500 mL). The organic phase was dried over MgSO₄ and evaporated to dryness to give an oily residue. The residue was suspended in diisopropylether (150 mL) and the resulting solid was filtered. The solid was washed with diisopropylether (100 mL) and dried under high vacuum at room temperature to give 3,4-methylenedioxybenzyl 4,6-O-benzylidene-2-O-(4-chlorobenzoyl)-3-O-(2,3,4,6-tetra-O-(4-chlorobenzyl)-α-D-galactopyranosyl)-β-D-galactopyranoside (9) (6.7 g, 47%) as a white powder.

R_{f} 0.50 (EtOAc/Hexane 1:1) MS (electrospray) C₆₂H₅₅Cl₅O₁₄ (1201.38) m/z (%)1221[M+Na]⁺ (80).

3,4-Methylenedioxybenzyl 4,6-O-benzylidene-3-O-(2,3,4,6-tetra-O-(4-chlorobenzyl)-α-D-galactopyranosyl)-β-D-galactopyranoside (10)

To a solution of sodium methoxide (280 mg, 10.4 mmol) in dry methanol (50 mL), 3,4-methylenedioxy-benzyl 4,6-O-benzylidene-2-O-(4-chlorobenzoyl)-3-O-(2,3,4,6-tetra-O-(4-chlorobenzyl)-α-D-galactopyranosyl)-β-D-galactopyranoside (9) (6.3 g, 5.2 mmol) in dry THF-MeOH 2:1 (150 mL) was added. The resulting mixture was stirred at 40° C. for 5 hours. The reaction mixture was cooled to 18° C. and neutralized (pH 7.0) with Aimberlite IR-120H⁺ cation exchange resin. The resin was filtered off and the filtrate evaporated to dryness to give an oily residue. The &rude product was suspended in hexane (200 mL), which was then vigorously stirred to break up the clumps. The suspension was-filtered and dried in vacuum at room temperature to give 3,4-methylenedioxybenzyl 4,6-O-benzylidene-3-O-(2,3,4,6-tetra-O-(4-chlorobenzyl)-α-D-galactopyranosyl)-β-D-galactopyranoside (10) (5.2 g, 93%) as a white powder.

R_{f} 0.30 (CH₂Cl₂/ethanol 50:1), MS (electrospray) m/z C₅₅H₅₂Cl₄O₁₃ (1062.83) m/z (%) 1098[M+K]⁺ (72)

3,4-methylenedioxybenzyl 3-O-(α-D-galactopyranosyl)-β-D-galactopyranoside (11)

To a suspension of Pd/C (10%) catalyst (220 mg) in a mixture of THF-EtOR-H₂O 6:2:1 (5 mL), a solution of 3,4-methylenedioxybenzyl 4,6-O-benzylidene-3-O-(2,3,4,6-tetra-O-(4-chlorobenzyl)-α-D-galactopyranosyl)-β-D-galactopyranoside (10) (200 mg, 0.19 mmol) in a mixture of THF-EtOH-H₂O 6:2:1 (5 mL) was added. The resulting suspension was shaken under a positive pressure (45 PSI) of hydrogen for 2.5 h. The reaction mixture was filtered through celite and the filtrate was concentrated under high vacuum at room temperature to a volume of approximately 15 mL. The resulting yellow solution was diluted with deionised water (50 mL) and neutralized (pH 7.0) with excess mixed bed resin (Amberlite-MB 1). The aqueous suspension was filtered and the filtrate was evaporated to dryness under high vacuum to give the crude product as a colourless residue. The crude product was purified by chromatography using CHCl₃-MeOH—H₂O 5:5:1 as the mobile phase to give 3,4-methylenedioxybenzyl 3-O-(α-D-galactopyranosyl)-β-D-galactopyranoside (11) (72 mg, 73%).

R_{f} 0.42 (CHCl₃/MeOH/H₂O 5:5:1) MS (electrospray) C₂₀H₂₈O₁₃ (476.43) m/z (%) 499[M+Na]⁺ (38), 477[M+H]⁺ (72)

3-O-(α-D-Galactopyranosyl)-D-galactopyranose (12)

A mixture of Pd(OH)₂ (20%) Pearlman's catalyst (0.7 g) and 3,4-methylenedioxybenzyl 4,6-O-benzylidene-3-O-(2,3,4,6-tetra-O-(4-chlorobenzyl)-α-D-galactopyranosyl)-β-D-galactopyranoside (10) (2.0 g, 1.9 mmol) in a mixture of THF-MeOH—H₂O 4:1:1 (30 mL) was shaken under a positive pressure (60 PSI) of hydrogen overnight. The reaction mixture was filtered through celite and the filtrate was neutralized with mixed-bed ion exchange resin (Amberlite-MB 1)/negative silver (I) nitrate test/. The reaction mixture was filtered and the filtrate was concentrated to dryness in vacuum at room temperature. The residue was taken up in deionised water (2 mL) and passed through a C18 Sep Pak cartridge eluting with milli-Q-water (30 mL). The filtrate was evaporated under reduced pressure to give 3-O-(α-D-galactopyranosyl)-D-galactopyranose (12) (560 mg, 86%) as a white solid foam.

TLC (CHCl₃-MeOH—H₂O 10:10:2) R_{f}=0.3, High performance anion exchange chromatography with pulsed amperometric detection /HPAE-PAD/ (4×250 mm Dionex CarbopaK-PA1 analytical column with guard column, 150 mM sodium hydroxide at 1 mL/min.) t_{R}=5.0 min., MS (electrospray) m/z 365 [M+Na]⁺.

R_{f} 0.30 (CHCl₃/MeOH/H₂O 5:5:1) MS (electrospray) C₁₂H₂₂O₁₁ (342.29) m/z (%) 406[M+Na+MeCN]⁺ (100), 365[M+Na]⁺ (62)

### EXAMPLE 4

### Preparation of 2-[2-(2-thiobiphenylcarbonyl)-ethoxy]ethyl 3-O-α-D-galactopyranosyl-β-D-galactopyranoside (23)

The synthesis of the reagents for this preparation and the preparation scheme itself are set out in Reaction Schemes 2 and 3 respectively.

Reagents for the Synthesis of 2-[2-(2-Thiobiphenyl-carbonyl)-ethoxy]ethyl 3-O-α-D-Galactopyranosyl-β-D-Galactopyranoside

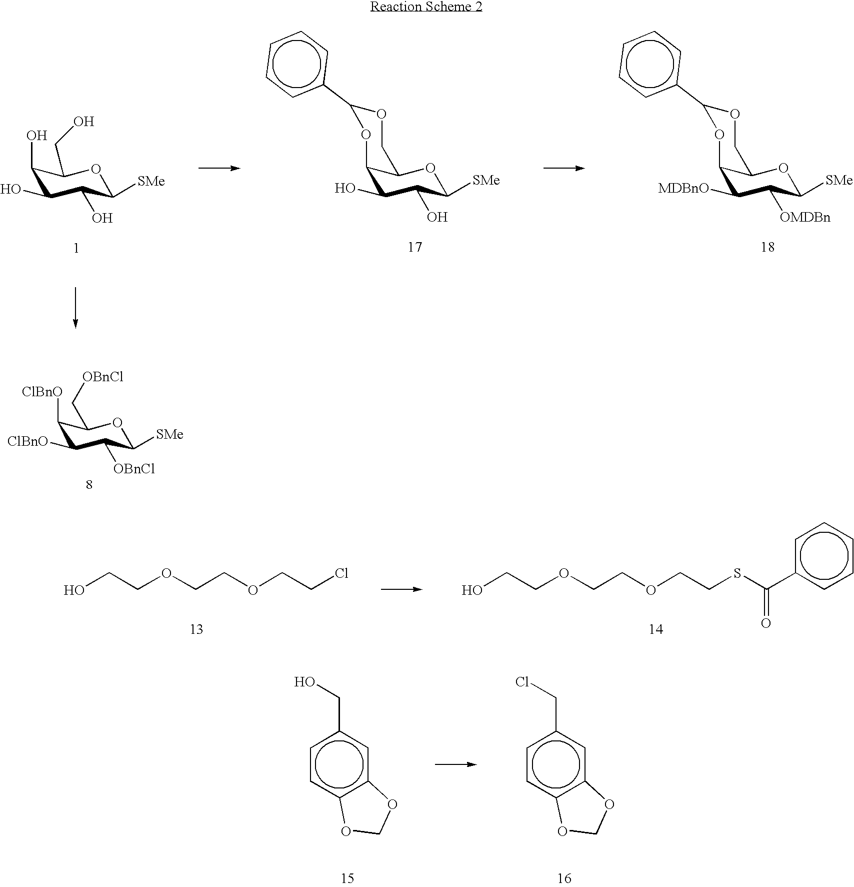

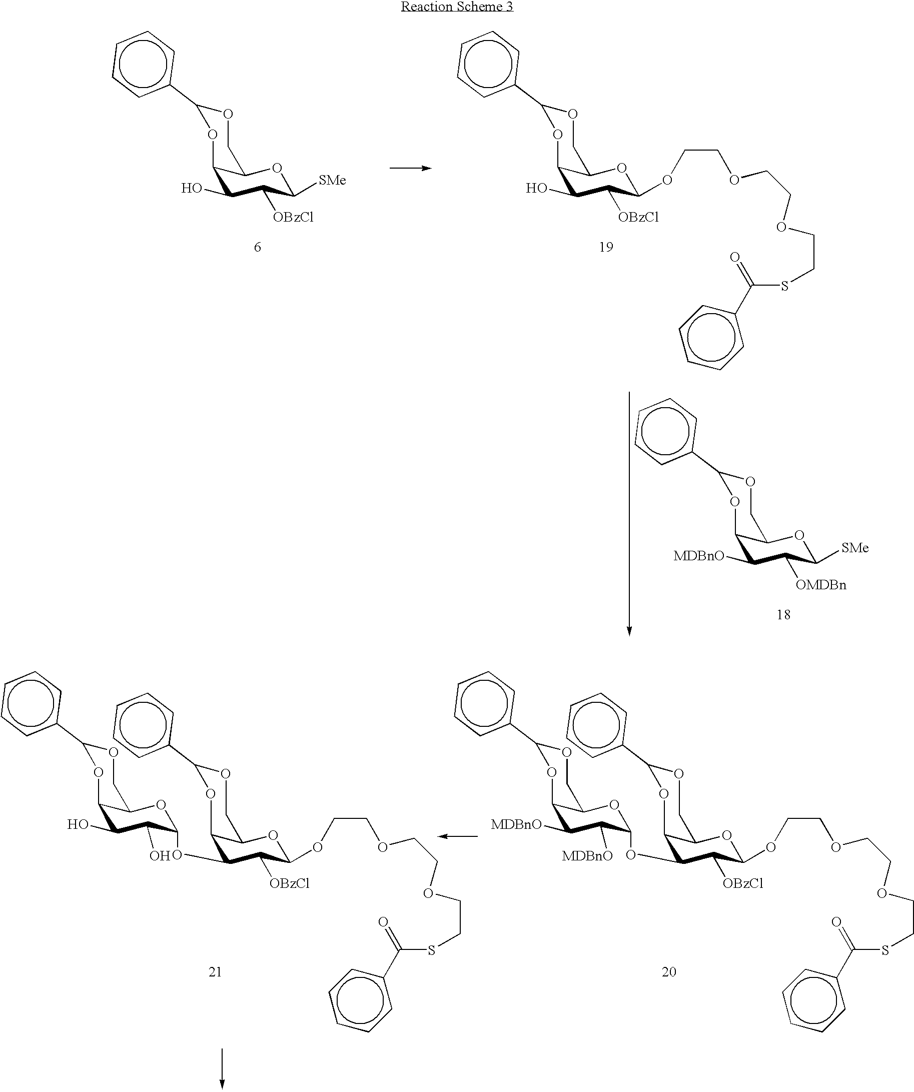

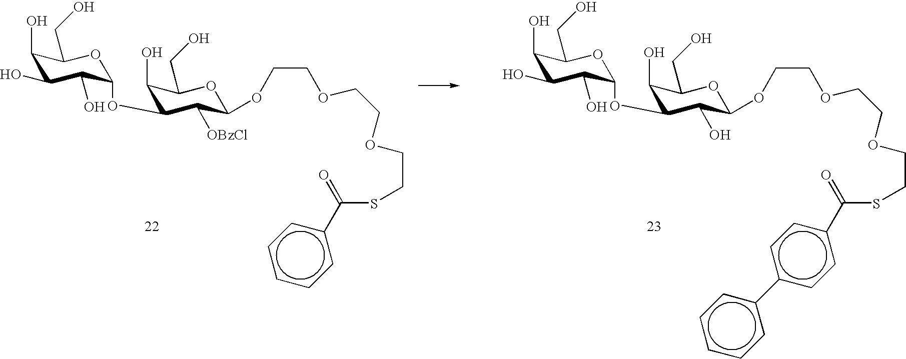

2-[2-(2-Thiobenzoyl)ethoxy]ethanol (14)

A mixture of 2-[2-(2-chloroethoxy)ethoxy]ethanol (13) (17.1 g, 101 mmol) and cesium thiobenzoate (38.24 g, 142 mmol) in dry DMF (200 ml) was stirred at 75° C. for 1.5 hours. The reaction mixture was cooled to room temperature and evaporated to dryness. The residue was taken up in diethylether (600 ml), washed three times with saturated NaHCO₃ solution (400 ml) and with water (500 ml). The organic phase was dried over MgSO₄ and evaporated to dryness to give 23 g of crude product. The crude residue was purified by chromatography using diethylether as the mobile phase to give 2-[2-(2-thiobenzoyl)ethoxy]ethanol (14) (18.75 g, 68%) as an orange syrup.

R_{f} 0.60 (diethylether/EtOH 19:1) MS (electrospray) C₁₃H₁₈O₄S (270.34) m/z (%) 293[M+Na]⁺ (62), 271[M+H]⁺ (100)

3,4-Methylenedioxybenzyl chloride (16)

A solution of 3,4-methylenedioxybenzyl alcohol (15) (50 g, 328.62 mmol) in CH₂Cl₂ (50 ml) was cooled to 0° C. and SOCl₂ (250 ml) added dropwise. The reaction mixture was stirred at 0° C. for 1 hour, at room temperature for 4 hours, then evaporated to dryness. The residue was purified by distillation under vacuum to give 3,4-methylenedioxybenzyl chloride (16) (49 g, 87%).

R_{f} 0.75 (CHCl₃/EtOAc 20:1)

Methyl 4,6-O-benzylidene-1-thio-β-D-galactopyranoside (17)

A mixture of methyl 1-thio-β-D-galactopyranoside (1) (23.6 g, 112 mmol), α,α-dimethoxytoluene (25.62 g, 168 mmol) and p-toluenesulphonic acid (100 mg) in MeCN (500 ml) was stirred at room temperature for 30 minutes. The-reaction mixture was neutralized with triethylamine (1 ml) and evaporated to dryness, followed by a co-evaporation with toluene. The residue was taken up in CH₂Cl₂ (250 ml), washed twice with brine (250 ml), dried over MgSO₄ and evaporated. The resulting white solid was crystallized from EtOH to give methyl 4,6-O-benzylidene-1-thio-β-D-galactopyranoside (17) (27.5 g, 82%).

R_{f} 0.32 (EtOAc) MS (electrospray) C₁₄H₁₈O₅S (298.36) m/z (%) 321[M+Na]⁺ (32), 299[M+H]⁺ (100)

Methyl 4,6-O-benzylidene-2,3-di-O-(3,4-methylenedioxy-benzyl)-1-thio-β-D-galactopyranoside (18)

A mixture of methyl 4,6-O-benzylidene-1-thio-β-D-galactopyranoside (17) (20 g, 66.80 mmol) and sodium hydride (95%) (4.80 g, 201.2 mmol) in dry DMF (350 ml) was stirred at 0° C. for 30 minutes, then 3,4-methylenedioxy-benzyl chloride (34.3 g, 201.2 mmol) (16) added in DMF (20 ml). The reaction mixture was stirred at room temperature overnight. Methanol (20 ml) was added and the reaction mixture was evaporated to dryness. The residue was taken up in CH₂Cl₂ (500 ml), washed twice with brine (500 ml), dried over MgSO₄ and evaporated. The residue was crystallized from 2-propanol (1 l) to give methyl 4,6-O-benzylidene-2,3-di-O-(3,4-methylenedioxybenzyl)-1-thio-β-D-galactopyranoside (18) (19 g, 50%).

R_{f} 0.62 (CHCl₃/EtOAc 20:1), MS (electrospray) C₃₀H₃₀O₉S (566.62) m/z (%) 589[M+Na]⁺ (100), 567[M+H]⁺ (25)

2-[2-(2-Thiobenzoyl)ethoxy]ethyl 4,6-O-benzylidene 2-O-(4-chlorobenzoyl)-β-D-galactopyranoside (19)

A mixture of methyl 4,6-O-benzylidene-2-O-(4-chlorobenzoyl)-1-thio-β-D-galactopyranoside (6) (10 g, 22.93 mmol), 0.2-[2-(2-thiobenzoyl)ethoxy]ethanol (13) (6.81 g, 25.22 mmol), powdered molecular sieves 4 Å (˜20 g) and dimethyl(methylthio)sulfonium tetrafluoroborate (7.0 g, 35.71 mmol) was stirred in dry 1,2-dichloroethane (100 mL) at 0° C. for 2 hours. The mixture was neutralized with triethylamine (10 mL), diluted with CH₂Cl₂ (300 mL) and filtered through celite. The filtrate was washed three times with saturated sodium bicarbonate solution (200 mL), dried over MgSO₄ and evaporated to dryness. The residue was suspended in diisopropylether (600 mL) and filtered. The resulting solid was crystallized from ethanol (50 ml), washed with diisopropylether (200 mL) and dried to give 2-[2-(2-thiobenzoyl)ethoxy]ethyl 4,6-O-benzylidene 2-O-(4-chlorobenzoyl)-β-D-galactopyranoside (19) (10 g, 66%) as a white powder.

R_{f} 0.30 (Diethylether/EtOAc 2:1), MS (electrospray) C₃₃H₃₅ClO₁₀S (659.15) m/z (%) 681[M+Na]⁺ (70), 659[M+H]⁺ (40)

2-[2-(2-Thiobenzoyl)ethoxy]ethyl 4,6-O-benzylidene-2-O-(4-chlorobenzoyl)-3-O-[4,6-O-benzylidene-2,3-di-O-(3,4-methylenedioxybenzyl)]-α-D-galactopyranosyl)-β-D-galactopyranoside (20)

A mixture of 2-[2-(2-thiobenzoyl)ethoxy]ethyl 4,6-O-benzylidene 2-O-(4-chlorobenzoyl)-β-D-galactopyranoside (19) (8.55 g, 12.99 mmol), methyl 4,6-O-benzylidene-2,3-di-O-(3,4-methylenedioxybenzyl)-1-thio-β-D-galactopyranoside (18) (8.00 g, 14.29 mmol), powdered molecular sieves 4A (20 g) and methyl trifluoromethanesulfonate (4.68 g, 28.58 mmol) was stirred in dry 1,2-dichloroethane (100 mL) at room temperature for 2 hours. The mixture was neutralized with triethylamine (4 mL), diluted with CH₂Cl₂ (200 mL) and filtered through celite. The filtrate was washed three times with saturated NaHCO₃ solution (200 mL), dried over MgSO₄ and evaporated to dryness. The residue was purified by chromatography using diethylether-EtOAc 2:1 as the mobile phase to give 7.5 g-of 2-[2-(2-thiobenzoyl)ethoxy]ethyl 4,6-O-benzylidene-2-O-(4-chlorobenzoyl)-3-O-[4,6-O-benzylidene-2,3-di-O-(3,4-methylenedioxybenzyl)]-α-D-galactopyranosyl)-β-D-galactopyranoside (20) (7.5 g, 50%) as a white solid foam.

R_{f} 0.55 (Diethylether/EtOAc 2:1), MS (electrospray) C₆₂H₆₁ClO₁₉S (1177.67) m/z (%) 1199[M+Na]⁺ (100), 1177 (21)

2-[2-(2-Thiobenzoyl)ethoxy]ethyl 4,6-O-benzylidene-2-O-(4-chlorobenzoyl)-3-O-(4,6-O-benzylidene-α-D-galactopyranosyl)-β-D-galactopyranoside (21)

A mixture of 2-[2-(2-thiobenzoyl)ethoxy]ethyl 4,6-O-benzylidene-2-O-(4-chlorobenzoyl)-3-O-[4,6-O-benzylidene-2,3-di-O-(3,4-methylenedioxybenzyl)]-α-D-galactopyranosyl)-β-D-galactopyranoside (20) (7.02 g, 5.97 mmol) and 2,3-dichloro-5,6-dicyano-1,4-benzoquinone (2.71 g, 11.93 mmol) in the mixture of CH₂Cl₂/H₂O 7:2 (70 ml) was stirred at room temperature for 1 hour. The reaction mixture was filtered, the filtrate was diluted with CHCl₃ (300 ml), washed twice with saturated NaHCO₃ solution (150 ml) and concentrated to dryness. The residue was taken up in hot diisopropylether (150 ml) and the solution was stirred at room temperature for 2 hours. The resulting suspension was filtered, then crystallized from EtOAc (40 ml). The mother liquid was purified by chromatography using diethylether-EtOAc 1:1 mixture as the mobile phase. The purified products were combined to give 2-[2-(2-thiobenzoyl)ethoxy]ethyl 4,6-O-benzylidene-2-O-(4-chlorobenzoyl)-3-O-(4,6-O-benzylidene-α-D-galacto-pyranosyl)-β-D-galactopyranoside (21) (3.69 g, 68%).

R_{f} 0.32 (Diethylether/EtOAc 2:1), MS (electrospray) C₄₆H₄₉ClO₁₅S (909.40) m/z (%) 931[M+Na]⁺ (35), 909[M+H]⁺ (100)

2-[2-(2-Thiobenzoyl)ethoxy]ethyl 2-O-(4-chlorobenzoyl)-3-O-α-D-galactopyranosyl-β-D-galactopyranoside (22)

A mixture of 2-[2-(2-thiobenzoyl)ethoxy]ethyl 4,6-O-benzylidene-2-O-(4-chlorobenzoyl)-3-O-(4,6-O-benzylidene-α-D-galactopyranosyl)-β-D-galactopyranoside (21) (3.5 g, 3.85 mmol) and p-toluenesulphonic acid (100 mg) in the mixture of acetonitrile-methanol 1:1 (350 ml) was stirred under reflux for 2 hours. The reaction mixture was evaporated to dryness then the residue was chromatographed using MeCN—H₂O 10:1 as the mobile phase to give 2-[2-(2-thiobenzoyl)ethoxy]ethyl 2-O-(4-chlorobenzoyl)-3-O-α-D-galactopyranosyl-β-D-galactopyranoside (22) (2.46 g, 87%).

R_{f} 0.42 (MeCN/H₂O 10:1), MS (electrospray) C₃₂H₄₁ClO₁₅S (733.13) m/z (%) 755[M+Na]⁺ (52), 733[M+H]⁺ (100)

2-[2-(2-Thiobiphenylcarbonyl)ethoxy]ethyl 3-O-α-D-galactopyranosyl-β-D-galactopyranoside (23)

A mixture of 2-[2-(2-thiobenzoyl)ethoxy]ethyl 2-O-(4-chlorobenzoyl)-3-O-α-D-galactopyranosyl-β-D-galactopyranoside (22) (210 mg, 0.287 mmol) and sodium methoxide (9 mg, 0.287 mmol) in dry methanol (15 ml) was stirred at 40° C. for 4 hours. The reaction mixture was cooled to room temperature and biphenylcarbonyl chloride (62.17 mg, 0.287 mmol) was added. After 30 minutes stirring at room temperature, the reaction mixture was evaporated to dryness. The residue was purified by chromatography using MeCN—H₂O 5:1 as the mobile phase to give 2-[2-(2-thiobiphenylcarbonyl)ethoxy]ethyl 3-O-α-D-galactopyranosyl-β-D-galactopyranoside (23) (120 mg, 62%).

R_{f} 0.35 (MeCN/H₂O 10:2), MS (electrospray) C₃₁H₄₂O₁₄S (670.73) m/z (%) 693[M+Na]⁺ (100), 671[M+H]⁺ (20)

### EXAMPLE 5

### Preparation of 2-Acetamido-2-Deoxy-4-O-[3-O-(α-D-Galactopyranosyl)-β-D-Galactopyranosyl]-D-Glucopyranose (28)

The general strategy for this preparation is set out in Reaction Scheme 4.

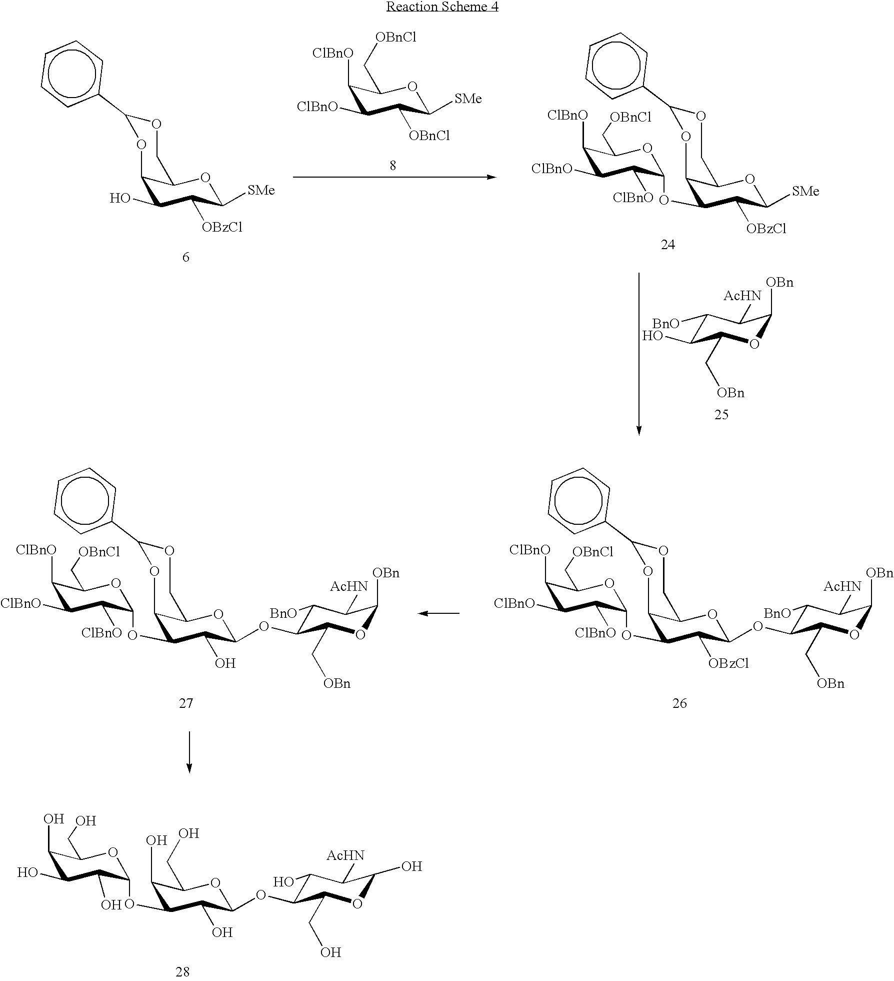

Methyl 4,6-O-benzylidene-2-O-(4-chlorobenzoyl)-3-O-(2,3,4,6-tetra-O-(4-chlorobenzyl)-α-D-galactopyranosyl)-1-thio-β-D-galactopyranoside (24)

A mixture of methyl 2,3,4,6-tetra-O-(4-chlorobenzyl)-thio-β-D-galactopyranoside (8) (3.9 g, 5.5 mmol), molecular sieves 4 Å (4 g) in dry THF (30 ml) was stirred at room temperature, then a solution of bromine (1.18 g, 6.66 mmol) in CH₂Cl₂ (5 ml) was added. The reaction mixture was stirred at room temperature for 10 minutes, then cyclohexene (1 ml) added. To the stirred reaction mixture methyl 4,6-O-benzylidene 2-O-(4-chlorobenzoyl)-β-D-galactopyranoside (6) (2.0 g, 3.7 mmol)was added then the suspension was cooled to −15° C. A solution of silver trifluoromethanesulphonate (1.4 g, 5.5 mmol) in dry THF (10 ml) was added dropwise under-nitrogen atmosphere in 15 minutes. The reaction mixture was kept at 0° C. overnight. The reaction mixture was neutralized with triethylamine (2 ml) and filtered. The filtrate was evaporated to dryness and the residue was taken up in CHCl₃ (300 mL). The solution was washed with saturated NaHCO₃ solution (3×300 mL). The organic phase was dried over MgSO₄ and evaporated to dryness to give an oily residue. The residue was chromatographed using diethylether-ethanol 20:1 as the mobile phase to give methyl 4,6-O-benzylidene-2-O-(4-chlorobenzoyl)-3-O-(2,3,4,6-tetra-O-(4-chloro-benzyl)-α-D-galactopyranosyl)-1-thio-β-D-galactopyranoside (24) (1.60 g, 40%).

R_{f} 0.30 (Diethylether), MS (electrospray) C₅₅H₅₁Cl₅O₁₁S (1097.33) m/z (%) 1117[M+Na]⁺ (100), 1095[M+H]⁺ (32)

Benzyl 2-acetamido-3,6-di-O-benzyl-2-deoxy-4-O-[4,6-O-benzylidene-2-O-(4-chlorobenzoyl)-3-O-(2,3,4,6-tetra-O-(4-chlorobenzyl)-α-D-galactopyranosyl)-β-D-galactopyranosyl)]-α-D-glucopyranoside (26)

A mixture of methyl 4,6-O-benzylidene-2-O-(4-chlorobenzoyl)-3-O-(2,3,4,6-tetra-O-(4-chlorobenzyl)-α-D-galactopyranosyl)-1-thio-β-D-galactopyranoside (24) (430 mg, 0.39 mmol), benzyl 2-acetamido-3,6-di-O-benzyl-2-deoxy-α-D-glucopyranoside (25) (300 mg, 0.59 mmol), molecular sieves 4 Å (5 g) and methyl trifluoromethane-sulphonate (97 mg, 0.59 mmol) in dry 1,2-dichloroethane (15 ml) was stirred at room temperature overnight. The reaction mixture was neutralized with triethylamine (2 ml) and filtered. The filtrate was diluted with CHCl₃ (100 ml) and was washed with saturated NaHCO₃ solution (2×100 mL). The organic phase was dried over MgSO₄ and evaporated to dryness to give an oily residue. The residue was chromatographed using diethylether-ethanol 25:1 as the mobile phase to give benzyl 2-acetamido-3,6-di-O-benzyl-2-deoxy-4-O-[4,6-O-benzylidene-2-O-(4-chlorobenzoyl)-3-O-(2,3,4,6-tetra-O-(4-chlorobenzyl)-α-D-galactopyranosyl)-β-D-galactopyranosyl)]-α-D-glucopyranoside (26) (300 mg, 50%).

R_{f} 0.33 (Diethylether/EtOH 25:1), MS (electrospray) C₈₃H₈₀Cl₅NO₁₇ (1540.83) m/z (%) 1560[M+Na]⁺ (100), 1538[M+H]⁺ (27)

Benzyl 2-acetamido-3,6-di-O-benzyl-2-deoxy-4-O-[4,6-O-benzylidene-3-O-(2,3,4,6-tetra-O-(4-chlorobenzyl)-α-D-galactopyranosyl)-β-D-galactopyranosyl)]-α-D-glucopyranoside (27)

To a solution of sodium methoxide (73 mg, 0.13 mmol) in dry methanol (10 mL), benzyl 2-acetamido-3,6-di-O-benzyl-2-deoxy-4-O-[4,6-O-benzylidene-20-(4-chlorobenzoyl)-3-O-(2,3,4,6-tetra-O-(4-chlorobenzyl)-α-D-galactopyranosyl)-β-D-galactopyranosyl)]-α-D-glucopyranoside (26) (300 mg, 0.19 mmol was added. The resulting mixture was stirred at 40° C. for 4.5 hours. The reaction mixture was kept at 0° C. for 1 hour and filtered. The solid precipitate was washed with-cold dry MeOH (10 ml) to give benzyl 2-acetamido-3,6-di-O-benzyl-2-deoxy-4-O-[4,6-O-benzylidene-3-O-(2,3,4,6-tetra-O-(4-chlorobenzyl)-α-D-galactopyranosyl)-β-D-galactopyranosyl)]-α-D-glucopyranoside (27) (190 mg, 67%) as a white powder.

R_{f} 0.35 (CHCl₃/MeOH 7:3), MS (electrospray) C₇₆H₇₇ClNO₁₆ (1402.27) m/z (%) 1423[M+Na]⁺ (100), 1401[M+H]⁺ (35)

2-Acetamido-2-deoxy-4-O-[3-O-(α-D-galactopyranosyl)-β-D-galactopyranosyl]-D-glucopyranose (28)

To a suspension of Pd/C (10%) catalyst (1.0 g), benzyl 2-acetamido-3,6-di-O-benzyl-2-deoxy-4-O-[4,6-O-benzylidene-3-O-(2,3,4,6-tetra-O-(4-chlorobenzyl)-α-D-galactopyranosyl)-β-D-galactopyranosyl)]-α-D-glucopyranoside (27) (190 mg, 0.13 mmol) and acetic acid (3 drops) was shaken under a positive pressure (45 PSI) of hydrogen for 4 hours. The reaction mixture was filtered through celite and the filtrate was neutralized (pH 7.0) with excess mixed bed resin (Amberlite-MB 1). The resin was filtered off and the filtrate was evaporated to dryness. The residue was taken up in milli-Q water (10 mL) and the resulting solution was filtered using a 0.22 μm filter. The filtrate was passed through a C-18 Sep-pak cartridge (1 g). The filtrate was evaporated-to dryness and the remaining solid was further dried over phosphorus pentoxide at room temperature under high vacuum to give 2-acetamido-2-deoxy-4-O-[3-O-(α-D-galactopyranosyl)-β-D-galactopyranosyl]-D-glucopyranose (28) (32 mg, 43%) as a white solid.

R_{f} 0.36 (CHCl₃/MeOH/H₂O 10:12:3), MS (electrospray) C₂₀H₃₅NO₁₆ (545.50) m/z (%) 568[M+Na]⁺ (100), 546[M+H]⁺ (52)

### EXAMPLE 6

### Alternative Synthesis of Compound (28)

Compound (28) may also be prepared using a different glucosamine acceptor, benzyl-6-O-benzoyl-3-O-benzoyl 1-2-acetamido-2-deoxy-x-D-glucopyranoside, using the strategy set out in Reaction Scheme 5. The acceptor can readily be prepared in high yield.

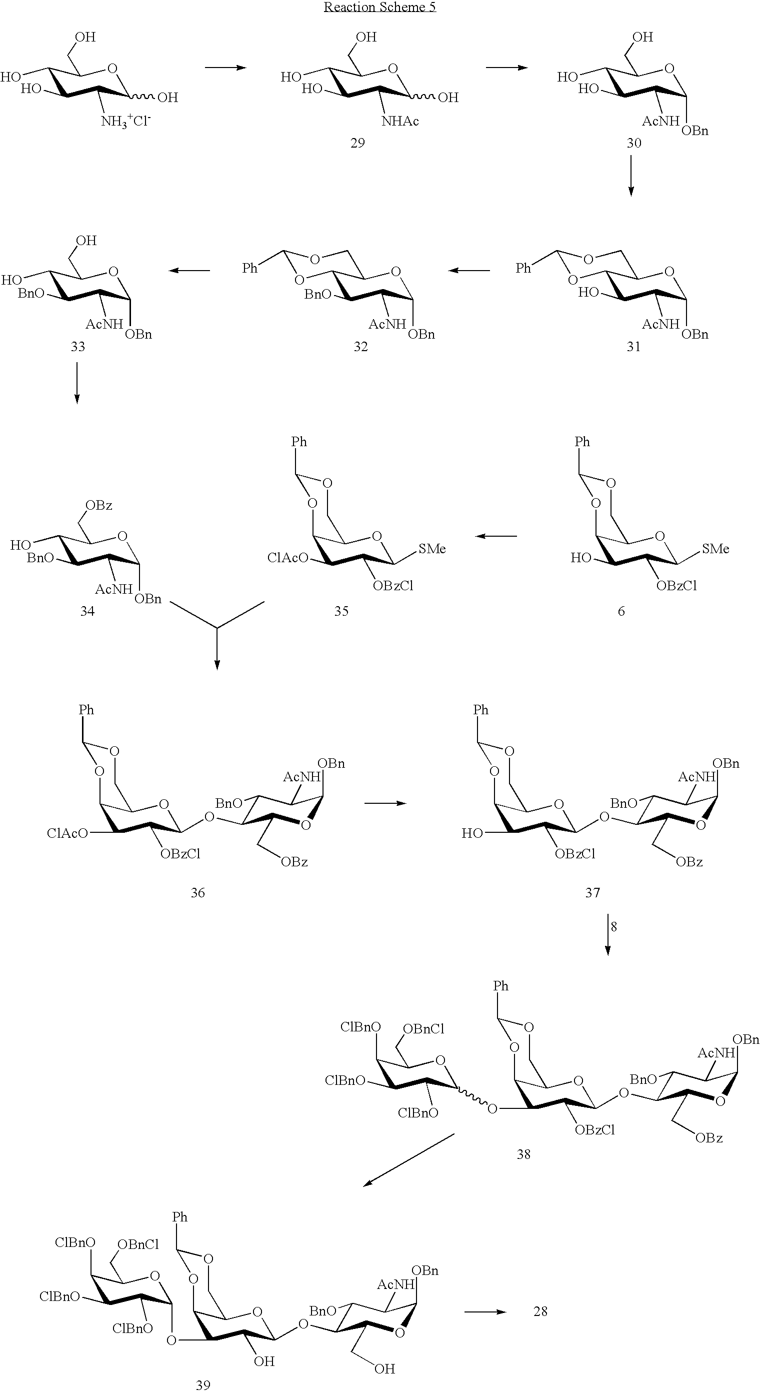

2-Acetamido-2-deoxy-D-glucopyranose (29)

Sodium (23.4 g, 1.02 mol) was reacted with dry methanol (1.6 L), then the resulting solution was cooled to 40° C. Glucosamine hydrochloride (200 g, 0.926 mol) was added to the solution and the reaction mixture was stirred vigorously for 5 minutes. The suspension was filtered in dry conditions. Acetic anhydride (140 mL, 1.48 mol) was added dropwise to the filtrate at 0° C. in 30 min. The resulting suspension was stirred at room temperature for another 30 minutes. The reaction mixture was diluted with ether (2 L), filtered and the solid product was dried to give 2-acetamido-2-deoxy-D-glucopyranose (29) (177 g, 86%).

Benzyl 2-acetamido-2-deoxy-α-D-glucopyranoside (30)

A mixture of 2-acetamido-2-deoxy-D-glucopyranose (29) (150 g, 0.68 mol), Amberlite IR 120 [H]⁺) ion exchange resin (150 g) in benzyl alcohol (1.25 L) was stirred at 80° C. for 3.5 hours. The reaction mixture was filtered. The filtrate was evaporated under reduced pressure at 90 C°. The residue was taken up in hot isopropanol (600 mL) and filtered. The filtrate was left to crystallize, the white crystalline solid was filtered off, washed twice with cold isopropanol (200 mL) and twice with ether (200 mL) to give 2-acetamido-2-deoxy-α-D-glucopyranoside (30)(56.2 g, 27%).

Benzyl 4,6-O-benzylidene-2-acetamido-2-deoxy-α-D-glucopyranoside (31)

Benzyl 2-acetamido-2-deoxy-α-D-glucopyranoside (30) (50 g, 0.16 mmol)-was dissolved in dry DMF (200 mL). Dry acetonitrile (100 mL), α,α-dimethoxytoluene (29 g, 0.19 mol, 1.2 eq) and p-toluenesulphonic acid (50 mg) was added to the DMF solution. The reaction mixture was stirred at 80° C. for 2 hours under vacuum (350 mbar); the product started to precipitate after 1 hour. The resulting suspension was cooled (60° C.) and the pH adjusted to 7 by addition of triethylamine. The suspension was cooled to 0° C., and cold methanol (500 mL) (−10° C.) was added slowly to the mixture. The product was filtered, washed with cold methanol (200 mL) then with cold ether (2×200 mL) to give benzyl 4,6-O-benzylidene-2-acetamido-2-deoxy-α-D-glucopyranoside (31) (48 g, 75%)

Benzyl 3-O-benzyl-4,6-O-benzylidene-2-acetamido-2-deoxy-α-D-glucopyranoside (32)

A suspension of sodium hydride (3.6 g, 0.15 mol, 1.2 eq) in dry DMF (25 mL) was cooled to 0° C., and a solution of benzyl 4,6-O-benzylidene-2-acetamido-2-deoxy-α-D-glucopyranoside (32) (50 g, 0.125 mol) in dry DMF (450 mL) was added dropwise in 30-minutes. The resulting solution was stirred at 0° C. for 30 minutes and benzyl bromide was added (25.66 g, 0.15 mol, 1.2 eq) dropwise at 0° C. (the product started to precipitate at the beginning of the addition of the benzyl bromide). The reaction mixture was stirred at room temperature for 45 minutes, cooled to 0° C. and dry methanol (25 mL) was added dropwise. The reaction mixture was diluted with cold ether (1 L) and the mixture was stirred for 30 minutes. The resulting suspension was filtered and washed three times with ether (400 mL) to give benzyl 3-O-benzyl-4,6-O-benzylidene-2-acetamido-2-deoxy-α-D-glucopyranoside (32) (62.0 g) as a white powder with quantitative yield.

Benzyl 3-O-benzyl-2-acetamido-2-deoxy-α-D-glucopyranoside (33)

A suspension of benzyl 3-O-benzyl-4,6-O-benzylidene-2-acetamido-2-deoxy-α-D-glucopyranoside (32) (50 g, 0.102 mol) in acetic acid (500 mL) and water (25 mL) was stirred at 110° C. for 45 minutes. The reaction mixture was concentrated under reduced pressure at 40 C°. The oily residue was taken up twice in toluene (200 mL) and concentrated. The residue was treated with di-isopropyl ether (250 mL) and the resulting suspension was stirred for 30 minutes. The white solid was filtered off, washed twice with cold ether (200 mL) to give benzyl 3-O-benzyl-2-acetamido-2-deoxy-α-D-glucopyranoside (33) (38.0 g, 93%).

Benzyl 6-O-benzoyl-3-O-benzyl-2-acetamido-2-deoxy-α-D-glucopyranoside (34)

A solution of benzoyl chloride (6.3 g, 0.045 mol, 1.2 eq) and imidazole (6 g, 0.09 mol, 2.4 eq) in dry 1,2-dichloroethane (150 mL) was stirred for 20 minutes at 5° C. The resulting suspension was filtered under dry conditions. The filtrate was added to a solution of benzyl 3-O-benzyl-2-acetamido-2-deoxy-α-D-glucopyranoside (33) (15 g, 37.6 mmol) in dry 1,2-dichloroethane (600 mL). The-reaction mixture was stirred at 90° C. for 48 hours and cooled to room temperature. The resulting suspension was filtered, washed twice with brine (300 mL), dried over MgSO₄ and concentrated. The residue was taken up in hot isopropanol (300 mL) and left to crystallize. The white crystalline solid was filtered off to give Benzyl 6-O-benzoyl-3-O-benzyl-2-acetamido-2-deoxy-α-D-glucopyranoside (34) (11.7 g, 62%).

Methyl 4,6-O-benzylidene-3-O-chloroacetyl-2-O-(4-chlorobenzoyl)-1-thio-β-D-galactopyranoside (35)

A mixture of methyl 4,6-O-benzylidene-2-O-(4-chlorobenzoyl)-1-thio-β-D-galactopyranoside (6) (10.0 g, 23 mmol) and 4-dimethylaminopyridine (3.40 g, 27.8 mmol) in dry 1,2-dichloroethane (100 mL) was stirred at 0° C., then chloroacetyl chloride (3.4 g, 27.8 mmol, 1.2 eq) was added dropwise to the solution. The reaction mixture was stirred at room temperature for 2.5 hours, then diluted with 1,2-dichloroethane (100 mL). The resulting solution was washed twice with saturated brine solution (100 ml), dried over MgSO₄ and concentrated to give methyl 4,6-O-benzylidene-3-O-chloroacetyl-2-O-(4-chlorobenzoyl)-1-thio-β-D-galactopyranoside (35) (10.2 g, 86%) as a white crystalline solid.

Benzyl 2-acetamido-6-O-benzoyl-3-O-benzyl-4-O-[4,6-O-benzylidene-3-O-chloroacetyl-2-O-(4-chlorobenzoyl)-β-D-galactopyranosyl]-2-deoxy-α-D-glucopyranoside (36)

To a mixture of benzyl 2-acetamido-6-O-benzoyl-3-O-benzyl-4-O-2-deoxy-α-D-glucopyranoside (34) (5 g, 9.9 mmol), methyl 4,6-O-benzylidene-3-O-chloroacetyl-2-O-(4-chlorobenzoyl)-1-thio-β-D-galactopyranoside (35) (5.71 g, 11.1 mmol, 1.12 eq) and Molecular sieves 4A (2.5 g) in dry 1,2-dichloroethane (300 mL), DMTST (5.75 g, 2.4 eq) was added under nitrogen. The reaction mixture was stirred at room temperature for 5 hours, then neutralized by addition of pyridine (5 mL). Acetic anhydride was added (2.5 mL) and the reaction mixture was stirred at room temperature for 0.5 hours. The resulting suspension was filtered through a bed of Celite. The filtrate was washed with a saturated solution of NaHCO₃ (200 mL), twice with brine (200 ml), dried over MgSO₄ and concentrated. The residue was taken up in DCM (25 mL) and diisopropyl ether (200 mL) was added. The resulting yellow precipitate was filtered off and washed twice with cold diisopropyl ether (100 mL). The solid was crystallized using a mixture of DCM (20 mL) and ether (25 mL) to give benzyl 2-acetamido-6-O-benzoyl-3-O-benzyl-4-O-[4,6-O-benzylidene-3-O-chloroacetyl-2-O-(4-chlorobenzoyl)-β-D-galactopyranosyl]-2-deoxy-α-D-glucopyranoside (36) (5.1 g, 0.55%) as a white crystalline solid.

Benzyl 2-acetamido-6-O-benzoyl-3-O-benzyl-4-O-[4,6-O-benzylidene-2-O-(4-chlorobenzoyl)-β-D-galactopyranosyl]-2-deoxy-α-D-glucopyranoside (37)

A mixture of benzyl 2-acetamido-6-O-benzoyl-3-O-benzyl-4-O-[4,6-o-benzylidene-3-O-chloroacetyl-2-O-(4-chlorobenzoyl)-β-D-galactopyranosyl]-2-deoxy-α-D-glucopyranoside (36) (0.5 g) and thiourea (303 mg) in THF (3 mL) and water (0.5 mL) was stirred at room temperature for 14 hours, then the reaction mixture was diluted with chloroform (100 mL). The resulting solution was washed twice with water (50 ml), dried over MgSO₄ and concentrated. The residue was purified by flash chromatography using DCM/EtOAc 1:1 as the mobile phase to give benzyl 2-acetamido-6-O-benzoyl-3-O-benzyl-4-O-[4,6-O-benzylidene-2-O-(4-chlorobenzoyl)-β-D-galactopyranosyl]-2-deoxy-α-D-glucopyranoside (37) (280 mg, 61%) as a white solid.

Benzyl 2-acetamido-6-O-benzoyl-3-O-benzyl-2-deoxy-4-O-[4,6-O-benzylidene-2-O-(4-chlorobenzoyl)-3-O-(2,3,4,6-tetra-O--(4-chlorobenzyl)-α,β-D-galactopyranosyl)-β-D-galactopyranosyl)]-α-D-glucopyranoside (38)

To a mixture of methyl 2,3,4,6-tetra-O-(4-chlorobenzyl)-1-thio-β-D-galactopyranoside (430 mg, 0.602 mmol), benzyl 2-acetamido-6-O-benzoyl-3-O-benzyl-4-O-[4,6-O-benzylidene-2-O-(4-chlorobenzoyl)-o-D-galactopyranosyl]-2-deoxy-α-D-glucopyranoside (37) (280 mg, 0.301 mmol) and molecular sieves 4 Å (300 mg) in dry 1,2-dichloroethane (3 mL), DMTST (300 mg, 1.2 mmol) was added. The reaction mixture was stirred at room temperature for 3 hours. The reaction mixture was neutralized with triethylamine (1 ml)., diluted with CHCl (50 mL) and filtered. The filtrate was then washed with-saturated NaHCO₃ solution (3×50 mL). The organic phase was dried over MgSO₄ and evaporated to dryness to give a solid foam. The residue was purified by chromatography using CHCl₃— EtOAc 1:1 as the mobile phase to give benzyl 2-acetamido-6-O-benzoyl-3-O-benzyl-2-deoxy-4-O-[4,6-O-benzylidene-2-O-(4-chlorobenzoyl)-3-O-(2,3,4,6-tetra-O-(4-chlorobenzyl)-α,β-D-galactopyranosyl)-β-D-galactopyranosyl)]-α-D-glucopyranoside (38) (325 mg, 70%, α/β=85/15).

Benzyl 2-acetamido-3-O-benzyl-2-deoxy-4-O-[4,6-O-benzylidene-3-O-(2,3,4,6-tetra-O-(4-chlorobenzyl)-α-D-galactopyranosyl)-β-D-galactopyranosyl)]-α-D-glucopyranoside (39)

To a solution of sodium methoxide (20 mg, 0.37 mmol) in dry methanol (2 mL), benzyl 2-acetamido-6-O-benzoyl-3-O-benzyl-2-deoxy-4-O-[4,6-O-benzylidene-2-O-(4-chlorobenzoyl)-3-O-(2,3,4,6-tetra-O-(4-chlorobenzyl)-α,β-D-galactopyranosyl)-β-D-galactopyranosyl)]-α-D-glucopyranoside (38) (190 mg, 0.12 mmol was added. The resulting mixture was stirred at 40° C. for 4 hours. The reaction mixture was cooled to room temperature and filtered. The solid precipitate was washed with cold dry MeOH (10 mL), followed by hexane (2×25 mL) to give benzyl 2-acetamido-3-O-benzyl-2-deoxy-4-O-[4,6-O-benzylidene-3-O-(2,3,4,6-tetra-O-(4-chlorobenzyl)-α-D-galactopyranosyl)-β-D-galactopyranosyl)]-α-D-glucopyranoside (39) (110 mg, 68%) as a white powder. TLC R_{f} 0.35 (EtOAc/CHCl₃ 7:3

2-Acetamido-2-deoxy-4-O-[3-O-(α-D-galactopyranosyl)-β-D-galactopyranosyl]-D-glucopyranose (28)

To a suspension of Pd/C (10%) catalyst (100 mg), benzyl 2-acetamido-3-O-benzyl-2-deoxy-4-O-[4,6-O-benzylidene-3-O-(2,3,4,6-tetra-O-(4-chlorobenzyl)-α-D-galactopyranosyl)-β-D-galactopyranosyl)]-α-D-glucopyranoside (39) (80 mg, 0.06 mmol) and acetic acid (3 drops) in THF-MeOH—H2O 5:1:1 (7 mL) was shaken under a positive pressure (60 PSI) of hydrogen overnight. The reaction mixture was diluted with milliQ water (30 mL), filtered through Celite and the filtrate was neutralized (pH 7.0) with excess mixed bed resin (Amberlite-MB 1). The resin was filtered off and the filtrate was evaporated to dryness. The residue was taken up in milli-Q water (5 mL) and the resulting solution was passed through a C-18 Sep-pak cartridge (1 g). The filtrate was evaporated to dryness and the remaining solid was further dried over phosphorus pentoxide at room temperature under high vacuum to give 2-acetamido-2-deoxy-4-O-[3-O-(α-D-galactopyranosyl)-β-D-galactopyranosyl]-D-glucopyranose (28). (20 mg, 53%) as a white solid.

R_{f} 0.36 (CHCl₃/MeOH/H₂O 10:12:3), MS (electrospray) C₂₀H₃₅NO₁₆ (545.50) m/z (%). 568[M+Na]⁺ (100), 546[M+H]⁺ (52)

### EXAMPLE 6

### Immobilization of 2-acetamido-2-deoxy-4-O-[3-O-(α-D-galactopyranosyl)-β-D-galactopyranosyl]-D-glucopyranose (28)

The following reaction scheme, Scheme 6, illustrates how a compound of the invention can be bound to a solid support, using two alternative linking groups. The second linking group is a dioxo compound, as discussed in our International patent application No. PCT/AU98/00808. It will be appreciated that other compounds of the invention can be linked to a solid support in a similar manner.

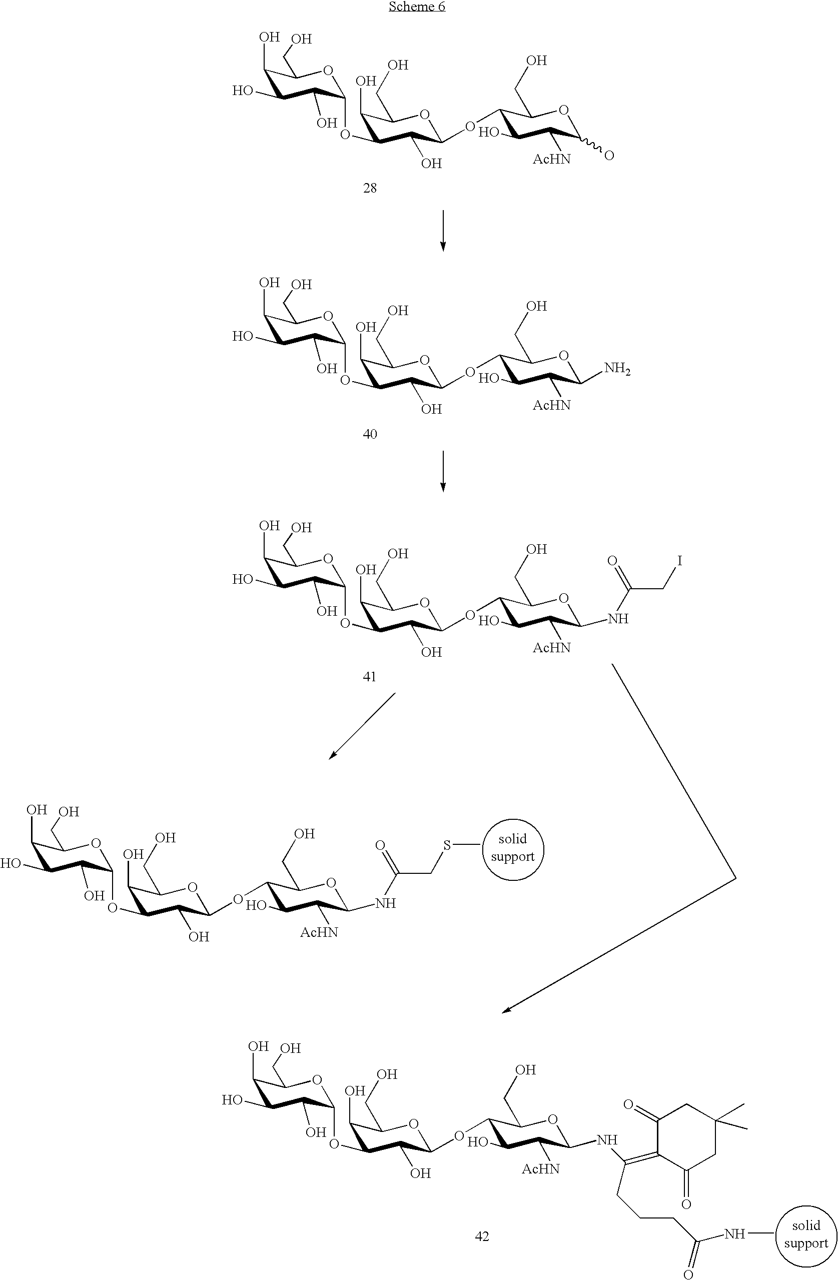

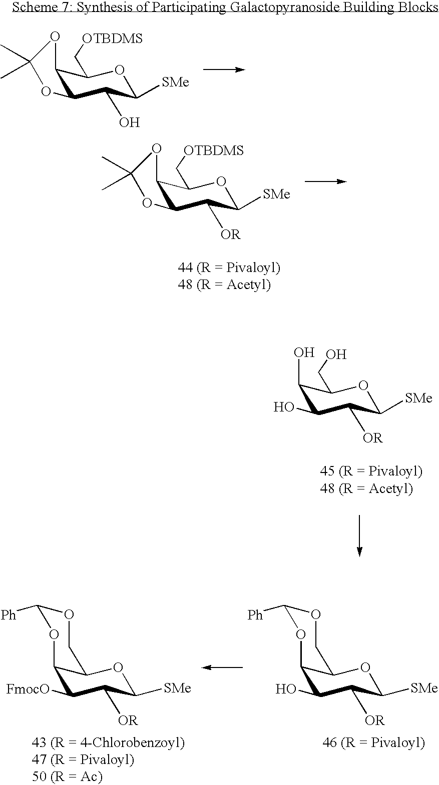

### EXAMPLE 7

### Synthesis of Methyl 4,6-O-benzylidene-2-O-(4-chlorobenzoyl)-3-O-fluorenylmethyl-oxycarbonyl-1-thio-β-D-galactopyranoside

Methyl 4,6-O-benzylidene-2-O-(4-chlorobenzoyl)-3-O-fluorenylmethyloxycarbonyl-1-thio-B D-galactopyranoside (43)

A suspension of methyl 4,6-O-benzylidene-2-O-(4-chlorobenzoyl)-1-thio-β-D-galactopyranoside 6 [20 g, 45.87 mmol] in 1,2-dichloroethane [200 mL was cooled to 0° C. To the cooled suspension was added DMAP [16.81 g, 138 mmol] followed by Fmoc-Cl [35.60 g, 137 mmol]]. The now solution was returned to ambient temperature and stirred for 2 hours. The reaction mixture was then diluted with Chloroform [200 mL, and washed with 5% citric acid solution [2×400 mL] and saturated brine solution [2×400 mL. The layers were separated and the organic layer dried over Na₂SO₄ followed by filtration and removal of the solvent in vacuo. The resulting residue was purified by column chromatography [20% ethylacetate/petroleum ethers v/v] to afford methyl 4,6-O-benzylidene-2-O-(4-chlorobenzoyl)-3-O-fluorenylmethyloxycarbonyl-1-thio-β-D-galactopyranoside 43 as a white foam [27.2 g, 90%]; R_{f}=0.22; ES-MS gave m/z (ion, relative intensity); ¹H NMR (CDCl₃) δ 7.88-7.07 (17H, aromatic), 6.01 (t, 1H, H-2), 5.79 (s, 1H, benzylidene) 5.36 (dd, 1H, H-3), 4.91 (d, 1H, J₁₋₂=8.5, H-1), 4.89 (d, 1H, H-4), 4.78 (dd, 1H, H-6ₐ), 4.67 (m, 2H, Fluorenyl-CH₂—), 4.52 (t, 1H, 9-fluorenylmethyne), 4.49 (dd, 1H, H-6_{b}), 4.14 (s, 1H, H-5), 2.29 (s, 3H, S—CH₃)

### EXAMPLE 8

### Synthesis of Methyl 4,6-O-benzylidene-3-O-fluorenylmethyloxycarbonyl-2-O-pivaloyl-1-thio-β-D-galactopyranoside

Methyl 6-O-tert-butyldimethylsilyl)-3,4-O-isopropylidene-2-O-(pivaloyl)-1-thio-β-D-galactopyranoside (44)

To a mixture 6-O-tert-butyldimethylsilyl-3,4-O-isopropylidene-1-thio-β-D-galactopyranoside [11.5 g, 31.59 mmol] and DMAP [5.5 g, 45.5 mmol] in 1,2-dichloroethane [40 mL) was added dropwise, 2,2,2-trimethylacetylchloride. The reaction was stirred for 2 hours then diluted with chloroform [100 mL] and washed with 10% citric acid solution [2×150 mL], saturated NaHCO₃ solution (2×150 mL] and saturated brine solution [2×150 mL]. The layers were separated and the organic layer dried over Na₂SO₄. The solvent was removed in vacuo to give an oily residue. The residue was purified by column chromatography (5% ethylacetate/petroleum ethers) to give a white foam, methyl 6-O-tert-butyldimethylsilyl-3,4-O-isopropylidene-2-O-pivaloyl-1-thio-β-D-galactopyranoside 44 (13.7 g, 97%]. R_{f}=0.75 (ethylacetate/petroleum ethers, 1:2, v/v); ¹H NMR (CDCl₃) δ 5.05 (dd, 1H, H-2), 4-29 (dd, 1H, H-4), 4.25 (d, 1H, J₁₋₂=10.12, H-1), 4.17 (dd, 1H, H-3), 3.93-3.84 (m, 3H, H-6ₐ, H-6_{b}, H-5), 2.16 (s, 3H, S—CH₃),

Methyl 2-O-pivaloyl-1-thio-β-D-galactopyranoside (45)

Methyl 6-O-tert-butyldimethylsilyl-2-O-pivaloyl-3,4-O-isopropylidene-1-thio-β-D-galactopyranoside 44 (3.34 g, 7.45 mmol] x, was dissolved in 25% acetonitrile/methanol [40 mL]. To the solution was added 4-toluenesulphonic acid [17 mg, 90.43 μmol], the solution was then stirred under refluxed for 3 hours. The reaction temperature was then reduced to 40° C. and left overnight. The reaction mixture was then concentrated and the residue azeotroped with toluene followed by diethylether to give a white residue. The residue was purified by column chromatography (10% acetonitrile/ethylacetate, v/v) to give a white solid, methyl 2-O-pivaloyl-1-thio-β-D-galactopyranoside 45 [2.19 g, 83%], R_{f}=0.20 (ethylacetate); ES-MS m/z (ion, relative intensity). 295 ([M+H]⁺, 100%); ¹H NMR (CDCl₃) δ 5.08 (dd, 1H, H-2), 4.39 (d, 1H, J₁₋₂=9.88 Hz, H-1), 4.13 (d, 1H, H-4), 4.01-3.92 (m, 2H, H-6ₐ, H-6_{b}), 3.72 (dd, 1H, H-3), 3.62 (dd, 1H, H-5), 2.21 (S, 3H, S—CH₃), 1.27 (s, 9H, t-butyl).

Methyl 4,6-O-benzylidene-2-O-pivaloyl-1-thio-β-D-galactopyranoside (46)

A mixture of methyl 2-O-(pivaloyl)-1-thio-β-D-galactopyranoside 45 [1.68 g, 5.71 mmol], α,α-dimethoxytoluene and 4-toluenesulphonic acid [10 mg, 43.19 mmol] was dissolved in acetonitrile [50 mL] and heated at 60° C. with stirring for 1 hour. The reaction was then allowed to return to ambient temperature, neutralised with 2 equivalents of triethylamine and concentrated under vacuum. The residue was taken up in chloroform [100 mL] and the organic layer washed with dilute brine [3:1; H₂O:Brine, 1×100 mL], saturated NaHCO₃ solution [1×100 mL], and saturated brine solution [1×100 mL]. The layers were separated and the organic layer dried over Na₂SO₄. The organic layer was concentrated and the residue purified by column chromatography (33% ethylacetate/petroleum ethers, v/v) to give methyl 4,6-O-benzylidene-2-O-pivaloyl-1-thio-β-D-galactopyranoside 46 [1.91 g, 87%]. R_{f}=0.63 (ethylacetate), ES-MS m/z (ion, relative intensity) 341 ([M+H]⁺, 100%); ¹H NMR (CDCl₃) δ 7.51 (m, 2H, aromatic) 7.41 (m, 3H, aromatic), 5.58 (s, 1H, CH-benzylidene), 5.24 (dd, 1H, H-2), 4.4 (dd, 1H, H-6ₐ), 4.39 (d, 1H, J₁₋₂=9.77, H-1), 4.29 (dd, 1H, H-4), 4.08 (dd, 1H, H-6_{b}), 3.8 (ddd, 1H, H-3), 3.60 (s, 1H, H-5), 2.26 (s, 3H, S—CH₃), 1.27 (s, 9H, t-butyl)

Methyl 4,6-O-benzylidene-3-O-fluorenylmethyloxycarbonyl-2-O-pivaloyl-1-thio-β-D-galactopyranoside (47)

Methyl 4,6-O-benzylidene-2-O-pivaloyl-1-thio-β-D-galactopyranoside 46 [1.90 g, 4.97 mmol] was dissolved in 1,2-dichloroethane (20 mL) and the resulting solution was cooled to 0° C. At this time DMAP [1.82 g, 14-92 mmol] and. Fmoc-Cl [3.87 g, 14.92 mmol] were added sequentially. The cold bath was then removed, and the reaction allowed to return to room temperature. The reaction was stirred at ambient temperature for 2 hours and then diluted with CHCl₃ [˜50 mL]. The reaction mixture was then washed with 5% citric acid solution [2×100 mL] and saturated brine solution [2×100 mL]. The layers were separated and the organic layer dried over Na₂SO₄. The solution was then filtered and concentrated to afford a yellow residue which was purified by column chromatography (20% ethylacetate/petroleum ethers v/v) to give methyl 4,6-O-benzylidene-3-O-fluorenylmethyloxycarbonyl-2-O-pivaloyl-1-thio-β-D-galactopyranoside 47 [2.74 g, 91%]; R_{f}=0.38 (25% ethylacetate/petroleum ethers v/v); ES-MS m/z (ion, intensity); ¹H NMR (CDCl₃) δ 7.78-7.25 (13H, aromatic), 5.61 (t, 1H, H-2), 5.57 (s, 1H, benzylidene), 4.97 (dd, 1H, H-3), 4.50 (d, 1H, H-4), 4.45 (d, 1H, J₁₋₂=9.10 hz, H-1), 4.47-4.33 (m, 2H, Fmoc-CH₂—), 4.25 (t, 1H, 9-fluorenylmethyne), 4.40, (dd, 1H, H-6ₐ) 4.08 (dd, *1H, H-6_{b}) 3.65 (s, 1H, H-5), 2.30 (s, 3H, S—CH₃), 1.20 (s, 9H, t-butyl)

### EXAMPLE 9

### Synthesis of Synthesis of Methyl 2-O-acetyl-4,6-O-benzylidene-3-O-fluorenylmethyloxycarbonyl-1-thio-β-D-galactopyranoside

Synthesis of Methyl 2-O-acetyl-6-O-tert-butyldimethylsilyl-3,4-O-isopropylidene-1-thio-β-D-galactopyranoside (48)

A mixture of methyl 6-O-tert-butyldimethylsilyl-3,4-O-isopropylidene-1-thio-β-D-galactopyranoside (3.0.0 g, 8.24 mmol) and 4-dimethylaminopyridine (2.42 g, 19.78 mmol) in dry 1,2-dichloroethane (750 ml) was stirred at room temperature. Acetyl chloride [1.05 mL, 14.84 mmol] was added dropwise to the solution over 15 minutes. The reaction stirred at room temperature for 2 hours at which time it was diluted with chloroform and washed with 10% citric acid solution [2×100 mL] saturated sodium hydrogen carbonate [2×100 mL] and finally with saturated brine solution [2×100 mL]. The layers were separated and the organic layer dried over Na₂SO₄. The solution was filtered and concentrated to afford a white residue which was purified by column chromatography (20% ethylacetate/petroleum ethers v/v) to afford methyl 2-O-acetyl-6-O-tert-butyldimethylsilyl-3,4-O-isopropylidene-1-thio-β-D-galactopyranoside 48 as a white solid [2.65 g, 79%]; R_{f}=0.43 (25% ethylacetate/petroleum ethers v/v)

Synthesis of Methyl 2-O-acetyl-1-thio-β-D-galactopyranoside (49)

2-O-Acetyl-6-O-tert-butyldimethylsilyl-3,4-O-isopropylidene-1-thio-β-D-galactopyranoside x was dissolved in 50% acetonitrile/methanol [50 mL] and heated at 60° C. To the stirred solution was added 4-toluenesulphonic acid [10 mg, 53.19 μmol] and the reaction was left for 4 hours. The reaction temperature was then reduced to 40° C. and left overnight. The reaction mixture was then concentrated and the residue crystallised from methanol to afford 2-O-acetyl-1-thio-β-D-galactopyranoside 49 as a white solid [1.26 g, 79%]; R_{f}=0.2 (25% acetonitrile/ethylacetate, v/v); ¹H NMR (d-MeOH) δ 3.95 (t, 1H, H-2), 3.27 (d, 1H, J₁₋₂=8.63, H-1), 2.92, 1H, H-4), 2.79-2.69 (m, 2H, H-6ₐ and H-6_{b}), 2.62 (t, 1H, H-3), 2.38 (m, 1H, H-5) 1.37 (s, 3H, S—CH₃), 1.31 (s, 3H, —C(O)CH₃)

Synthesis of Methyl 2-O-acetyl-4,6-O-benzylidene-3-O-fluorenylmethyloxycarbonyl-1-thio-β-D-galactopyranoside (50)

2-O-Acetyl-1-thio-β-D-galactopyranoside 49 was dissolved in acetonitrile [20 mL] and heated to 60° C. To the stirred solution was added α,α-dimethoxytoluene [1.09 g, 7.10 mmol] and 4-toluenesulphonic acid [10 mg, 53.19 μmol]. The reaction was stirred for 2 hours and then allowed to return to room temperature. The reaction was neutralised with 2 equivalents of triethylamine and evaporated to dryness. The residue was taken up in chloroform and washed with dilute brine [1×100 mL], saturated sodium hydrogencarbonate solution [1×100 mL] and saturated brine solution [1×100 mL]. The layers were separated and the organic layer dried over Na₂SO₄. The solution was filtered and concentrated. The residue was washed successively with petroleum ethers, and the resulting white solid then suspended in toluene and any remaining water azeotroped under co-evaporation. The residue from the previous step was suspended in 1,2-dichloroethane [20 mL] and cooled to 0° C. To the stirred suspension at 0° C. was added 4,4-dimethylaminopyridine [1.62 g, 13.23 mmol] and Fmoc-Cl [3.42 g, 12.23 mmol]. The now solution was allowed to return to room temperature and stirred for 1 hour. At this time the reaction was diluted with chloroform and washed with 5% citric acid solution [2×75 mL] and saturated brine solution [2×75 mL]. The layers were then separated and the organic layer dried over Na₂SO₄. The solution was filtered and the solvent removed in vacuo to give a yellow oily residue which was purified by column chromatography (33% ethylacetate/petroleum ethers v/v) to give methyl 2-O-acetyl-4,6-O-benzylidene-3-O-fluorenylmethyloxycarbonyl-1-thio-β-D-galactopyranoside 50 [2.19 g, 82%] R_{f}=0.2 (33% ethylacetate/petroleum ethers, v/v); ¹H NMR (CDCl₃) δ 7.78-7.24 (13H, aromatic), 5.60 (t, 1H, H-2), 5.55 (s, 1H, benzylidene), 4.88 (dd, 1H, H-2), 4.50 (d, 1H, H-4), 4.55-4.38 (m, 4H, H-1, Fmoc-CH₂, H-6ₐ), 4.28 (t, 1H, 9-fluorenyl-methyne), 4.06 (dd, 1H, H-6_{b}), 3.63 (s, 1H, H-5), 2.29 (s, 3H, S—CH₃), 2.1 (s, 3H, —C(O)CH₃)

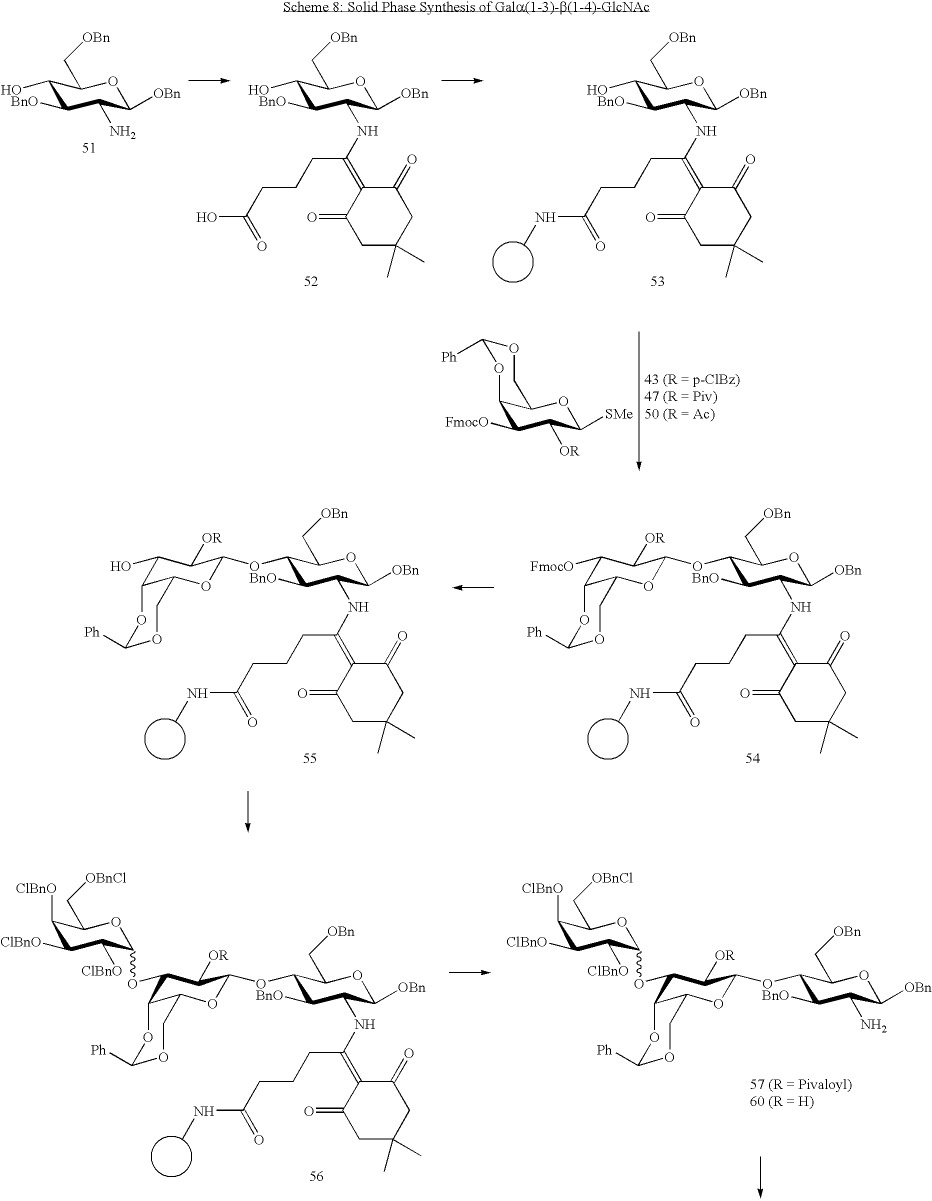

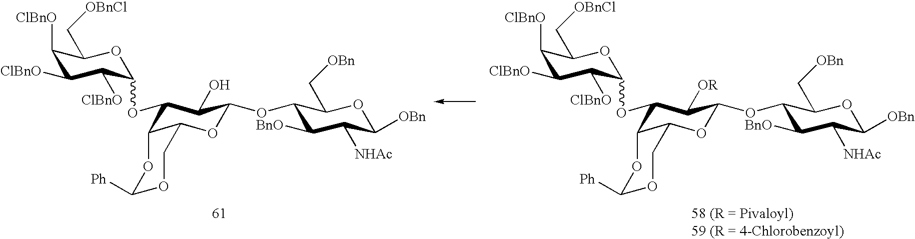

### EXAMPLE 10

### Synthesis of a Partially Protected Resin-Linker-Sugar Conjugate

Benzyl 3,6-di-O-benzyl-2-deoxy-2-amino-β-D-glucopyranoside (51)

To a solution benzyl of 3,6-di-O-benzyl-2-deoxy-2-phthalimido-β-D-glucopyranoside [6.20 g, 10.71 mmol] in ethanol [100 mL], was added hydrazine hydrate [6.2 mL, 55%/H₂O] and water [5 mL]. The solution was refluxed overnight and then allowed to return to ambient temperature. The solution was filtered, the solvent removed in vacuo, and the residue taken up in CHCl₃ [200 mL]. The Chloroform suspension was filtered, the filtrate dried over Na₂SO₄ and concentrated under reduced pressure to give a pure clear oil, benzyl 3,6-Di-O-benzyl-2-deoxy-2-amino-β-D-glucopyranoside 51 [4.7 g, 97%); R_{f}=0.5 (Acetonitrile), ES-MS gave m/z (ion, relative intensity): 450 ([M+H]⁺, 100%); ¹H NMR (CDCl₃) δ 7.43-7.30 (m 15H, aromatic), 5.00-4.60 (6H, 3CH₂—C₆H₅), 4.38 (d, 1H, J₁₋₂=7.92 Hz, H-1), 3.85-3.75 (m, 3H, H-6ₐ, H-6_{b}, H-3), 3.53 (ddd, 1H, H-5), 3.38 (dd, 1H, H-3), 2.92 (dd, 1H, H-2).

Benzyl 3,6-Di-O-benzyl-2-deoxy-2-N-(6-(4,4-dimethyl-2,6-dioxocyclohexylidene)-pentanoic acid-6-yl)-β-D-glucopyranoside (52)

To a solution of Benzyl 3,6-Di-O-benzyl-2-deoxy-2-amino-β-D-glucopyranoside 51 [4.70 g, 10.47 mmol] in ethanol [100 mL], was added 6-hydroxy-6-(4,4-dimethyl-2,6-dioxocyclohexylidene)-pentanoic acid [5.32 g, 20.93 mmol] followed by the addition of triethylamine [1.5 mL, 10.69 mmol]. The reaction mixture was heated overnight at 60° C. and then allowed to return to room temperature. The reaction mixture was concentrated and the residue taken up in chloroform [200 mL]. The organic layer was washed with a solution of 0.3N HCl [2×200 mL] and saturated Brine solution [1×200 mL]. The organic layer was dried over Na₂SO₄ and concentrated to give a pale yellow residue. The residue was purified by column chromatography with ethylacetate-petroleum ethers-acetic acid, 5:15:0.4 to give benzyl 3,6-Di-O-benzyl-2-deoxy-2-N-(6-(4,4-dimethyl-2,6-dioxocyclohexylidene)-pentanoic acid-6-yl)-β-D-glucopyranoside 52 [6.09 g, 85%]. R_{f}=0.10 (ethylacetate-petroleum ethers-acetic acid, 5:15:0.4), ES-MS m/z (ion, relative intensity): 686.5 ([M+H]⁺, 100%)

Coupling of Benzyl 3,6-Di-O-benzyl-2-deoxy-2-N-(6-(4,4-dimethyl-2,6-dioxocyclohexylidene)-pentanoic acid-6-yl)-β-D-glucopyranoside to MBHA resin (0.7 mmol/g) (53)

In a 200 mL peptide reaction vessel MBHA resin [11.86 g, 8.30 mmol] was swollen in a minimum of dry N,N-dimethylformamide (DMF). A DMF [50 mL] solution was made of Benzyl 3,6-Di-o-benzyl-2-deoxy-2-N-(6-(4,4-dimethyl-2,6-dioxocyclohexylidene)-pentanoic acid-6-yl)-β-D-glucopyranoside 52 [6.09 g, 8.90×mmol], diisopropylethylamine (DIPEA) [3.11 mL, 17.8 mmol] and O-Benzotriazole-1-yl-N,N,N′,N′-tetramethyluroniumhexa-fluorophosphate (HBTU) [3.37 g, 8.9 mmol] which was then added to the reaction vessel. The vessel was sealed and shaken overnight. Ninhydrin assay indicated that the reaction was greater than 99.4% complete, the reaction was stopped, and the resin was washed with DMF [4×100 mL], 50% DCM/MeOH [4×100 mL] and DCM [4×100 mL]. The resin was dried under house vacuum for 4 hours and then dried under high vacuum overnight. Yield of resin 53 was [17.15 g, 98.6% by weight].

Synthesis of Benzyl 2-acetamido-3,6-di-O-benzyl-2-deoxy-4-O-[4,6-O-benzylidene-2-O-pivaloyl-3-O-(2,3,4,6-tetra-O-(4-chlorobenzyl)-α-D-galactopyranosyl)-β-D-galactopyranosyl)]-β-D-glucopyranoside (58)

Under an atmosphere of nitrogen, resin 53 (300 mg, 141 μmol], 4,6-O-benzylidene-3-O-fluorenylmethyloxycarbon-yl-2-O-pivaloyl-1-thio-β-D-galactopyranoside 47 [557 mg, 846 μmol] and powdered molecular sieves 4 Å [600 mg], were suspended in dichloromethane [3 mL], followed by the addition of methyl trifluoromethanesulphonate [95.7 μL, 846 μmol]. The reaction vessel was sealed and the reaction mixture agitated for five hours at ambient temperature. The resin was then washed with DMF [3×20 mL], 50% MeOH/DCM [3×20 mL] and DCM [3×20 mL]. The resin was then floated in DCM to separate the resin from any remaining sieves. Resin 54 was collected and dried under house vacuum for 1 hour. The resin was then treated with a 20% triethylamine/DMF solution for 25 mins followed by workup as above. Resin 55 was dried under hi-vacuum overnight. Under an atmosphere of nitrogen the resin was then combined with methyl 2,3,4,6-tetra-O-(4-chlorobenzyl)-1-thio-β-D-galactopyranoside 8 [600 mg, 846 μmol], powdered molecular sieves 4 Å[800 mm] and dichloromethane [4 mL], followed finally by the addition of methyl trfluoromethanesulphonate [95.74 μL, 846 μmol]. The reaction vessel was sealed and the reaction mixture agitated at ambient temperature for five hours. The resin was then washed as standard and collected and dried on a sintered funnel. In a reaction vessel resin 56 was then combined with a 5% hydrazine hydrate (55%/H₂O)/DMF [5 mL] solution and agitated at ambient temperature for 4 h. The DMF solution was filtered from the resin and the resin then further washed with DMF [7 mL3. The filtrates were combined and the solvent removed in vacuo. The residue was taken up in minimal dichloromethane and passed through a plug of silica (eluent; DCM, TLC: CH₂Cl₂:MeOH, 20:0.3). The combined fractions were concentrated, residue 57 was then taken up in 1,2-dichloroethane [3 mL] and reacted with acetylchloride [46 μL, 648 μmol] in the presence of DMAP [84 mg, 684 μmol] for three hours at ambient temperature. The reaction was diluted with chloroform [20 mL] and washed with saturated citric acid solution [2×20 m], saturated sodium hydrogen carbonate solution (2×20 mL] and saturated brine solution [2×20 mL]. The organic layer was separated, dried over Na₂SO₄ and concentrated to give a white solid residue. The residue was purified by column chromatography (0.5% MeOH/DCM, v/v) to give 2-acetamido-3,6-di-O-benzyl-2-deoxy-4-O-[4,6-O-benzylidene-2-O-pivaloyl-3-O-(2,3,4,6-tetra-O-(4-chlorobenzyl)-α-D-galactopyranosyl)-β-D-galactopyranosyl)]-β-D-glucopyranoside 58 (213 mg, 76.3%). R_{f}=0.57 (66% ethylacetate/petroleum ethers, v/v), ES-MS m/z (ion, intensity) 1486.29 ([M+H]⁺ 100%)

In a cognate experiment to experiment 58, compound 47 was substituted with compound 43 (the experiment employing resin 53 (425 mg, 0.199 mmol/g)), to afford 2-acetamido-3,6-di-O-benzyl-2-deoxy-4-O-[4,6-O-benzylidene-2-O-(4-chlorobenzoyl)-3-O-(2,3,4,6-tetra-O-(4-chlorobenzyl)-α-D-galactopyranosyl)-β-D-galactopyranosyl)]-β-D-glucopyranoside 59 (96 mg, 34%), R_{f}=0.23 (1.64% methanol/dichloromethane, v/v), ES-MS m/z (ion, intensity) 1543.29 ([M+H]⁺ 100%)

In a further cognate experiment to experiment 58, compound 47 was substituted with compound 50 to afford 2-amino-3,6-di-O-benzyl-2-deoxy-4-O-[4,6-O-benzylidene-3-O-(2,3,4,6-tetra-O-(4-chlorobenzyl)-α-D-galactopyranosyl)-β-D-galactopyranosyl)]-β-D-glucopyranoside 60, R_{f}=0.5 (1.96% methanol/dichloromethane, v/v), ES-MS m/z (ion, intensity) 1360.73 ([M+H]⁺ 100%)

Synthesis of 2-Acetamido-3,6-di-O-benzyl-2-deoxy-4-O-[4,6-O-benzyli-dene-3-O-(2,3,4,6-tetra-O-(4-chlorobenzyl)-α-D-galactopyranosyl)-β-D-galactopyrano-syl)]-β-D-glucopyranoside (61)

2-Acetamido-3,6-di-O-benzyl-2-deoxy-4-O-[4,6-O-benzyli-dene-2-O-pivaloyl-3-O-(2,3,4,6-tetra-O-(4-chlorobenzyl)-α-D-galactopyranosyl)-β-D-galactopyranosyl)]-β-D-glucopyranoside 58 [288 mg, 188 μmol] was suspended in a solution of NaOMe/MeOH [0.13M, 10 mL] to which was added acetonitrile [5 mL]. The reaction was heated at 70° C. until TLC indicated that the reaction had gone to completion (4-5 days). The reaction mixture was then concentrated and taken up in dichloromethane [20 mL] and washed with 10% citric acid solution [2×20 mL] and saturated brine solution [2×20 mL]. The organic layer was separated, dried over Na₂SO₂ and the solvent removed in vacuo to provide a solid white residue. The residue was purified by preparative thin layer chromatography (eluent: 13% Acetone/DCM) to give 2-Acetamido-3,6-di-O-benzyl-2-deoxy-4-O-[4,6-O-benzyli-dene-3-O-(2,3,4,6-tetra-O-(4-chlorobenzyl)-α-D-galactopyranosyl)-β-D-galactopyranosyl)]-β-D-glucopyranoside 61 [189 mg, 69%]. R_{f} 0.24 (1.47% MeOH/DCM); ES-MS m/z (ion, intensity) 1403.29 ([M+H]⁺, 100%)

Synthesis and Immobilisation of Gal-α-(1-3)-Gal-β-(1-4)-GlcNAc-Linker Conjugate.

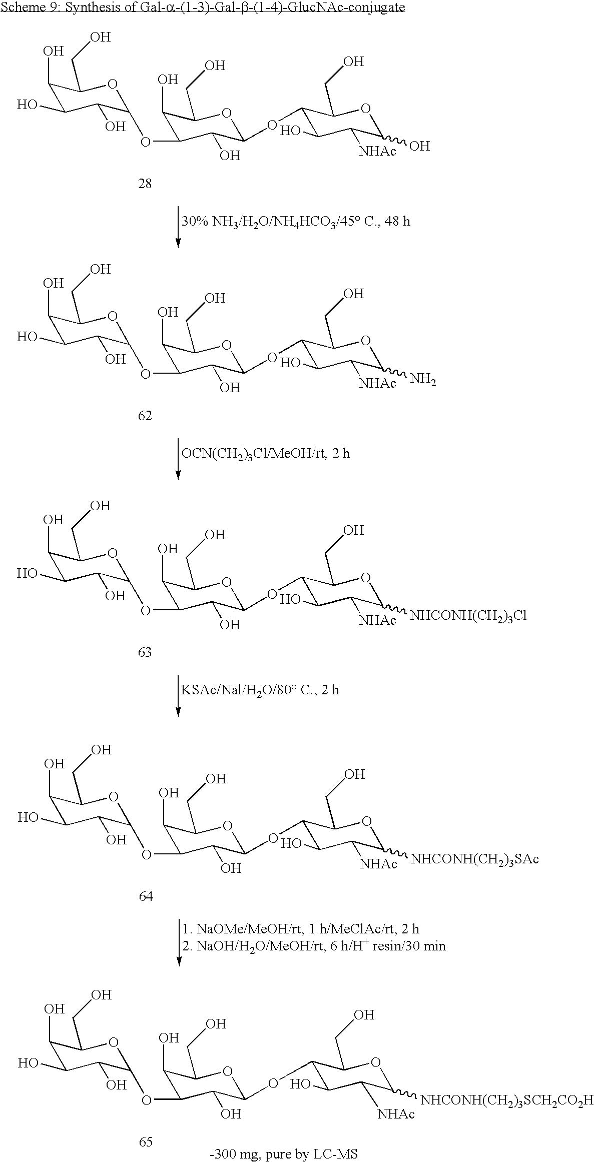

### EXAMPLE 11

### Synthesis of Sugar-Linker Conjugate

2-Acetamido-2-deoxy-4-O-[3-O-(α-D-galactopyranosyl)-β-D-galactopyranosyl]-D-glucopyranosylamine (62)

A solution of 2-Acetamido-2-deoxy-4-O-[3-O-(α-D-galactopyranosyl)-β-D-galactopyranosyl]-D-glucopyranose (1 g, 1.8 mmol) 28 and ammonium bicarbonate (0.15 g, 1.9 mmol) in 30% aqueous ammonia (20 mL) was left to stir at 40° C. for 48 h. The reaction mixture was then freeze dried to give 62 (1.0 g, ˜80% yield by tlc) as a white solid.

Tlc R_{f} 0.2 (AcN:water, 3:1)

1-N-(3-chloropropyl)-1-N′-ureido-2-acetamido-2-deoxy-4-O-[3-O-(α-D-galactopyranosyl)-β-D-galactopyranosyl]-D-glucopyranoside (63)

To a solution of 62 (0.35 g, 6.5 mmol) in methanol (5 mL), was added, 3-chloropropylisocyanate (0.1 g, 0.84 mmol). The reaction mixture was then left to stir at room temperature overnight. The reaction contents was evaporated to dryness and the remaining residue was dissolved in water* (˜3 mL) and loaded on to a C-18 Sep-pack column (5 g). The column was eluted** with water (50 mL) followed by 25% methanol in water (50 mL). The methanol fractions were combined and evaporated to dryness giving pure 63 (350 mg, ˜80% yield) as a white solid.

Tlc R_{f} 0.6 (AcN:water, 3:1)

M+H found 664

HPLC Rₜ 4.0 and 4.5 min for α/β anomers (linear gradient:

5% AcN to 20% AcN over

15 min, C-18 column)

1-N-(3-acetoxythiopropyl)-1-N′-ureido-2-acetamido-2-deoxy-4-O-[3-O-(α-D-galactopyranosyl)-β-D-galactopyranosyl]-D-glucopyranoside (64)

A mixture of 63 (0.2 g, 0.30 mmol), sodium iodide (0.1 g, 0.67 mmol) and potassium thioacetate (0.2 g, 1.74 mmol) in water (10 mL) was left to stir at 80° C. for 2 h. The reaction mixture was then cooled to room temperature and concentrated to 5 ml. The concentrate was loaded on to a C-18 Sep-pack column (5 g) which was then eluted with water (100 mL) followed by 25% methanol in water (100 mL). The methanol fractions were combined and evaporated to dryness to give pure 64 (0.18 g, ˜85% yield) as a white solid.

Tlc R_{f} 0.6 (AcN:water, 3:1)

M+H found 703

HPLC Rₜ 5.5 and 6.0 min for α/β anomers (linear gradient:

5% AcN to 20% AcN over

15 min, C-18 column)

1-N-[3-(methyl carboxymethythio)-propyl]-1-N′-ureido-2-acetamido-2-deoxy-4-O-[3-O-(α-D-galactopyranosyl)-β-D-galactopyranosyl]-D-glucopyranoside (65)

To a solution of sodium methoxide (14 mg, 0.26 mmol) in methanol (3 mL), was added 64 (110 mg, 0.24 mmol). The, reaction mixture was stirred at room temperature for 20 min and then methyl bromoacetate (50 mg, 0.30 mmol) was added. The resultant mixture was left to stir at room temperature for 2 h. The reaction mixture was quenched with acetic acid (200 μL) and then evaporated to dryness. The residue was dissolved in water (2 mL) and loaded on to a C-18 Sep-pack column (5 g). The column was eluted with water (50 ml) followed by 50% methanol in water (50 mL). The methanol fractions were combined and evaporated to dryness giving 65 (100.8 mg, 90% yield) as a white solid.

Tlc R_{f} 0.65 (AcN:water, 3:1)

M+H found 734, M+Na found 755

1-N-[3-(carboxymethylthio)-propyl]-1-N′-ureido-2acetamido-2-deoxy-4-O-[3-O-(α-D-galactopyranosyl)-β-D-galactopyranosyl]-D-glucopyranoside (66)

A solution of 65 (300 mg, 0.41 mmol) and potassium hydroxide (30 mg, 0.53 mmol) in 30% aqueous methanol (15 mL) was left to stir at room temperature for 4 h. The reaction mixture was diluted to 50 mL with methanol and then neutralised with IR-120H⁺ resin. The suspension was then filtered and the filtrate evaporated to dryness leaving 66 (295 mg, 100% yield) as a white solid.

Tlc R_{f} 0.30 (AcN:water, 3:1)

M+H found 719

Notes

*Milli-Q-Water was used at all times

**Flow rate was one drop/sec at all times

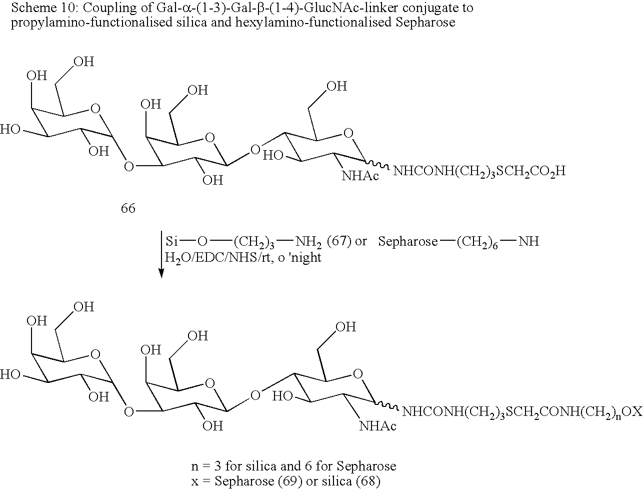

### EXAMPLE 12

### Immobilisation of Gal-α-(1-3)-Gal-β-(1-4)-GlucNAc-Linker Conjugate

Preparation of 0.3 mmol propylamido-Fmo-Ala-functionalised Silica (67)

To a mixture of FMOC-Ala (2.65 g, 8.5 mmol) and HBTU (3.23 g, 8.5 mmol) in dry DMF (20 mL), was added DIPEA (1.1 g, 8.5 mmol). The mixture was shaken for 2 min and then left to stand for 15-min. The mixture was then added to a suspension of propylamino functionalised silica* (17 g) in dry DMF (20 mL). The resultant mixture was shaken end over end for 18 h at room temperature. The mixture was filtered and the silica washed with DMF (3×100 mL) followed by methanol (3×100 mL). The resin was resuspended in a mixture of methanol (100 mL) and acetic anhydride (50 mL) and then shaken for 2 h (negative ninhydrin test after this time). The suspension was filtered and the silica was then washed with methanol (4×100 mL) and dried. The loading of FMOC-Ala was found to be 0.3 mmol per gram** of silica *Silica was washed with DIPEA prior to coupling. **FMOC-Ala loading was quantitated by cleaving (20% piperidine in DMF) a known quantity of FMOC-Ala capped silica and determining the concentration from the UV absorption of the cleavage product at 290 nm against a standard curve.

Coupling of 66 to propylamido-Ala-functionalised Silica (68)

FMOC-Ala modified silica from above was cleaved by the standard method (20% piperidine in DMF, rt, 20 min) to give the corresponding free amino (˜0.3 mmol loading) functionalised silica. This was then used for the trisaccharide couplings described below.

Loading 1, ˜20 mg of F per gram of Ala-capped Silica:

To NHS (235 mg, 2.08 mmol), was added a solution of 66 (100 mg, 0.139 mmol) and EDC.HCl (2.15 g, 11.2 mmol) in water (10 mL). The resulting solution was added to a suspension of Ala-capped silica (5 g) in water (˜10 mL). The suspension was left to shake at room temperature for 3 h, at which time no trisaccharide was present in the filtrate, by tlc. The suspension was then drained, washed with water (4×50 ml), dilute sodium bicarbonate solution (3×50 ml) and again with water (3×50 ml). The silica was then resuspended in methanol/acetic anhydride (30 ml, 3:1) and left to shake for 1 h (negative ninhydrin test after this time). The suspension was then drained and the silica washed with methanol (4×50 ml) to give the trisaccharide capped silica.

Loading 2, ˜5.0 mg of 66 per gram of Ala-capped silica:

66 (25 mg, 0.034 mmol), NHS (100 mg, 0.884 mmol), EDC.HCl (1.2 g, 6.25 mmol),

and Ala-capped silica (5 g).

Prepared as described for loading 1.

Loading 3, ˜0.5 mg of 66 Per Gram of Ala-capped Silica:

66 (2.5 mg, 0.0034 mmol), NHS (30 mg, 0.265 mmol), EDC.HCl (130 mg, 0.677 mmol),

and Ala-capped silica (5 g).

Prepared as described for loading 1.

Coupling of 66 to hexylamino-functionalised Sepharose (EAH Sepharose 4B) (69)

Loading, ˜3.5 to 6.0 mg of 66 per mL of EAH Sepharose:

EAH Sepharose (5 mL) was washed with water (3×50 ml) and then suspended in water (5 ml). To the suspension a solution of 66 (94 mg, 0.131 mmol), EDC.HCl (1.55 g, 8.10 mmol) and NHS (290 mg, 2.57 mmol) in water (15 mL) was added. The reaction mixture was left to shake overnight at room temperature. Tlc of the filtrate showed no 66 present after this time. The reaction contents were drained and the resin was washed with water (3×50 mL). The modified Sepharose was then stored as a concentrated suspension in 5% ethanol in water (5 mL).

It will be apparent to the person skilled in the art that while the invention has been described in some detail for the purposes of clarity and understanding, various modifications and alterations to the embodiments and methods described herein may be made without departing-from the scope of the inventive concept disclosed in this specification.

References cited herein are listed on the following pages, and are incorporated herein by this reference.

### REFERENCES

Augé, C. and Veyrières, A., J. C. S. Perkin I, 1979 1825-1832

Boriello, S. P., J. Med. Microb., 1990 33 207-215

Burakoff, R., Zhao, L., Celifarco, A. J. et al, Gastroenterology, 1995 109 348-354

Castex, P., Jouvert, S., Bastide, M. and Corthier, G. J. Med. Microbiol., 1994 40 102-109

Chacon-Fuertes, M. E. and Martin-Lomas, M. Carbohydrate Res., 1975 43 51-56

Eglow, R. et al. J. Clin. Invest., 1992 90 822-829

Garegg, P. J. and Oscarson, S. Carbohydrate Research, 1985 136 207-213

Good, H., Cooper, D. K. C. et al. Transplant. Proc., 1992 24 559

Ichiro, Matsuo., Hiroshi, Fujimoto., Megumi, Isomura. and Katsumi, Ajisaka., Biorganic & Medicinal Chemistry Letters, 1997 7 (3) 255-258

Krivan, H. C., Clark, G. F., Smith, D. F. and Wilkins, T. D. Infect. Immun., 1986 53 573-581

Lemieux, R. U. and Driguez, H., Journal of the American Chemical Society, 1975 97(14) 469-475

Matsuo, Ichiro; Fujimoto, Hiroshi; Isomura, Megumi and Ajisaki, Katsumi Bioorganic & Medicinal Chemistry Letters, 1997 7(3) 255-258

Milat, M-L., Zollo, P. A. and Sinay, P. Carbohydrate Research, 1982 100 263-271

Nilsson, K. G. I. Tetrahedron Letters, 1997 38 (1) 133-136

Schaubach, R., Hemberger, J. and Kinzy, W. Liebigs Ann. Chem., 1991 607-614

Simon, P. M., DDT 1 (12) December 1996

Sinaÿ, P. and Jacquinet, J. C. Tetrahedron, 1979 35 365-371

Smith, J. A. et al. J. Med. Microb., 1997 46 953-958

Sujino, Keiko., Malet, Charles., Hindsgaul, Ole. and Palcic, Monica M. Carbohydrate Research, 1998 305 483-489

Takeo, Ken'ichi and Maeda, Hideaki J. Carbohydrate Chemistry, 1988 7(2) 309-316

Tong Zhu and Geert-Jan Boons J. Chem. Soc., Perkin Trans.I, 1998 857-861

Torres, J., Jennische, E., Lange, S. and Lonnroth, I., Gut, 1990 31 781-785

Vic, G., Chuong Hao Tran, Scigelova, M. and Crout, D. H. G. Chem. Commun., 1997 169-170

1. A glucosamine compound of general formula I: 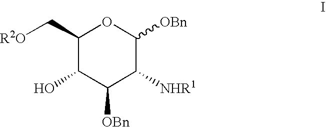
in which R¹ is h or acetyl and R² is benzyl or 4-chlorobenzoyl,
with the proviso that when R² is benzyl, R¹ is not acetyl.

2. A protected monosaccharide building block of general formula II: 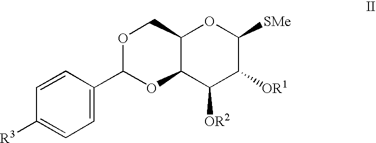
in which R³ is methoxy or methyl;
R¹ is H, benzoyl, pivaloyl, 4-chlorobenzoyl, acetyl, chloroacetyl, levulinoyl, 4-methylbenzoyl, benzyl, 3,4-methylenedioxybenzyl, 4-methoxybenzyl, 4-chlorobenzyl, 4-acetamidobenzyl, or 4-azidobenzyl; and
R² is H, Fmoc, benzoyl, pivaloyl, 4-chlorobenzoyl, acetyl, chloroacetyl, levulinoyl, 4-methylbenzoyl, benzyl, 3,4-methylenedioxybenzyl, 4-methoxybenzyl, 4-chlorobenzyl, 4-acetamidobenzyl, or 4-azidobenzyl.

3. A protected monosaccharide building block according to claim 2, in which
R³ is H, R¹ is benzoyl, pivaloyl, 4-chlorobenzoyl, acetyl, chloroacetyl, levulinoyl, benzyl, 3,4-methylene-dioxybenzyl, 4-methoxybenzyl, 4-chlorobenzyl, 4-acetamidobenzyl, or 4-azidobenzyl, and
R² is Fmoc, benzoyl, 4-chlorobenzoyl, acetyl, chloroacetyl, levulinoyl, 4-methylbenzoyl, benzyl, 3,4-methylenedioxybenzyl, 4-methoxybenzyl, 4-chlorobenzyl, 4-acetamidobenzyl, or 4-azidobenzyl,
with the provisos that
(a) when R¹ is acetyl, R² is not chloroacetyl or acetyl, and vice versa;
(b) when R² is levulinoyl, R¹ is not benzoyl, and vice versa; and
(c) when R¹ is benzoyl, R² is not benzoyl, and vice versa.

4. A protected monosaccharide building block according to claim 2 or claim 3, in which R² is Fmoc, and R¹ is benzoyl, pivaloyl, 4-chlorobenzoyl, acetyl, chloroacetyl, levulinoyl, 4-methylbenzoyl, benzyl, 3,4-methylene-dioxybenzyl, 4-methoxybenzyl, 4-chlorobenzyl, 4-acetamidobenzyl, or 4-azidobenzyl.

5. A protected monosaccharide building block according to any one of claims 2 to 4, in which the compound is of general formula III: 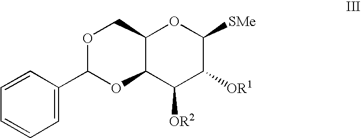
in which R¹ is pivaloyl, benzoyl, 4-chlorobenzoyl, 4-methoxybenzyl, or 3,4-methylenedioxybenzyl, and
R² is H, Fmoc, 4-chlorobenzoyl, acetyl, chloroacetyl, levulinoyl, 4-methoxybenzyl, or 3,4-methylenedioxybenzyl, with the proviso that if R¹ is benzoyl, R² is not levulinoyl.

6. A protected monosaccharide building block according to claim 5, in which the compound is a galactopyranoside, R¹ is 4-chlorobenzoyl, pivaloyl or acetyl, and R² is FMoc or H.

7. A protected monosaccharide building block according to claim 5, in which R¹ is 4-chlorobenzoyl and R² is chloroacetyl.

8. A protected monosaccharide building block according to claim 5, in which both R¹ and R² are 3,4-methylenedioxybenzyl.

9. A galactopyranoside compound of general formula IV: 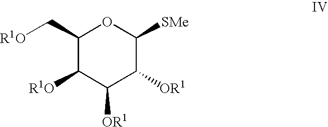
in which each R¹ is independently 4-chlorobenzyl, 4-azidobenzyl, 4-N-acetamidobenzyl, 4-methylbenzyl, 3,4-methylenedimethoxybenzyl, or 2-nitrobenzyl.

10. A galactopyranoside according to claim 9, in which each R¹ is 4-chlorobenzyl.

11. A polyethyleneglycol (PEG)-linked monosaccharide of general formula V: 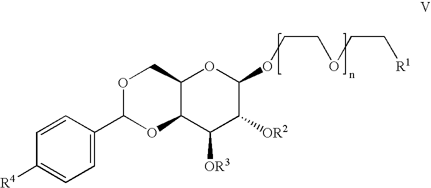
in which n is an integer from 1-5;
R¹ is a linking group or a group suitable for the formation of a covalent linkage;
R² is acetyl, 4-chlorobenzoyl, levulinoyl, pivaloyl, chloroacetate, benzoyl, 4-methybenzoyl;
R³ is H, Fmoc, benzoyl, pivaloyl, 4-chlorobenzoyl, acetyl, chloroacetyl, levulinoyl, 4-methylbenzoyl, 3,4-methylenedioxybenzyl, 4-methoxybenzyl, 4-acetamidobenzyl, or 4-azidobenzyl; and
R⁴ is methoxy, H, or methyl.

12. A polyethyleneglycol (PEG)-linked monosaccharide according to claim 11, in which R¹ is selected from the group consisting of halogen, azido, carboxylic acid, thiol, hydroxyl, thioester, xanthate, amido, and dithiocarbamate.

13. A PEG-linked monosaccharide according to claim 11 or claim 12, in which n is 2, R¹ is thiobenzoate or thiobiphenylcarbonyl, R² is 4-chlorobenzoyl, R³ is H, and R⁴ is H.

14. A compound of general formula VI: 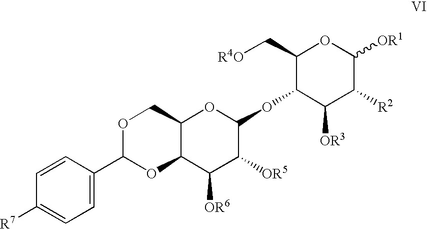
in which R⁷ is H, methoxy or methyl;
R¹ is aryl, substituted aryl, benzyl, substituted benzyl, alkyl, substituted alkyl, PEG, or substituted PEG;
R² is acetamido or amino;
R³ and R⁴ are independently benzyl, substituted benzyl, silylether or acyl;
R⁵ is 4-chlorobenzoyl, benzoyl, pivaloyl, acetyl, levulinoyl or 4-methylbenzoyl; and
R⁶ is a substituted or unsubstituted pyranosyl or furanosyl sugar, H, Fmoc, acetyl, chloroacetyl, levulinoyl, 3,4-methylenedioxybenzyl, 4-methoxybenzyl, 4-acetamidobenzyl, or 4-azidobenzyl.

15. A compound according to claim 14, in which the anomeric configuration of the the glucosamine moiety is a; R³ is benzyl, R⁴ is benzoyl and R⁷ is H, R² is optionally acetamido, amino, or N-phthalimido, R⁵ is optionally 4-chlorobenzoyl, benzoyl, pivaloyl, acetyl, levulinoyl or 4-methylbenzoyl, and R⁶ is a substituted or unsubstituted pyranosyl or furanosyl sugar, H, Fmoc, acetyl, chloroacetyl, levulinoyl, 3,4-methylenedioxybenzyl, 4-methoxybenzyl, 4-acetamidobenzyl, or 4-azidobenzyl.

16. A compound according to claim 14, in which the anomeric configuration of the the glucosamine moiety is β; R¹ is benzyl and R⁷ is H, R² is acetamido, amino, or N-phthalimido; R³ and R⁴ are independently benzyl, substituted benzyl, silylether or acyl; R⁵ is 4-chlorobenzoyl, benzoyl, pivaloyl, acetyl, levulinoyl or 4-methylbenzoyl, and R⁶ is a substituted or unsubstituted pyranosyl or furanosyl sugar, H, Fmoc, acetyl, chloroacetyl, levulinoyl, 3,4-methylenedioxybenzyl, 4-methoxybenzyl, 4-acetamidobenzyl, or 4-azidobenzyl.

17. A compound according to claim 14, in which the anomeric configuration of the the glucosamine moiety is α; R¹, R³, and R⁴ are benzyl or substituted benzyl, and R⁷ is H, R² is acetamido, amino, or N-phthalimido, R⁵ is pivaloyl, 4-chlorobenzoyl, benzoyl, or levulinoyl, and R⁶ is a substituted or unsubstituted pyranosyl or furanosyl sugar, H, Fmoc, acetyl, chloroacetyl, levulinoyl, 3,4-methylenedioxybenzyl, 4-methoxybenzyl, 4-acetamidobenzyl, or 4-azidobenzyl, with the proviso that when R³ and R⁴ are benzyl, R⁵ is not acetyl or benzoyl.

18. A compound according to claim 14, in which the anomeric configuration of the the glucosamine moiety is β; R¹ is benzyl, R² is amino or acetamido, R³ and R⁴ are benzyl, R⁵ is 4-chlorobenzoyl, pivaloyl or acetyl, R⁶ is Fmoc or H, and R⁷ is H.

19. A compound according to claim 14, in which the anomeric configuration of the the glucosamine moiety is a; R¹ is benzyl, R² is acetamido, R³ is benzyl, R⁴ is benzoyl or benzyl, R⁵ is 4-chlorobenzoyl, R⁶ is H or 4-chloroacetyl and R⁷ is H.

20. A compound according to claim 14, in which the compound is a trisaccharide of General Formula VII: 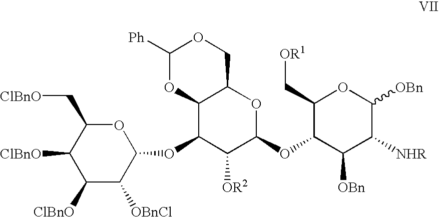
in which R is H or acetyl; R¹ is hydrogen, benzyl, benzoyl or p-chlorobenzoyl; and R² is hydrogen, 4-chloro-benzoyl, acetyl, benzoyl or pivaloyl.

21. A compound according to claim 20, in which the anomeric configuration of the reducing end of the trisaccharide is α, R is acetyl, R¹ is benzoyl, 4-chlorobenzoyl or H, and R² is 4-chlorobenzoyl or H.

22. A compound according to claim 20, in which the anomeric configuration of the reducing end of the trisaccharide is β, R is acetyl or H, R¹ is benzyl, and R² is H, 4-chlorobenzoyl, pivaloyl or acetyl.

23. A compound of general formula VIII: 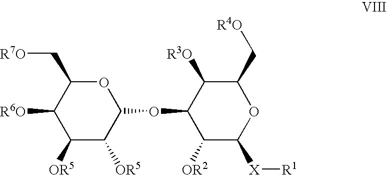
in which R⁵, R⁶ and R⁷ are independently H, 4-chlorobenzyl, 4-methoxybenzyl, 4-methylbenzyl, 4-acetamidobenzyl, azidobenzyl or 3,4-methylenedioxybenzyl;
X is O, S, or N;
R¹ is alkyl, substituted alkyl, aryl, substituted aryl, PEG or substituted PEG;
R² is levulinoyl, 4-chlorobenzoyl, benzoyl, 4-methylbenzoyl, acetyl or pivaloyl; and
R³ and R⁴ either combine to form a benzylidene ring, which may optionally be substituted at the 4 position by methyl or methoxy, or R³ and R⁴ are independently H, benzyl or substituted benzyl.

24. A compound according to claim 23, in which R⁵ is 4-chlorobenzyl, 4-methoxybenzyl, 4-methylbenzyl, 4-acetamidobenzyl, azidobenzyl or 3,4-methylenedioxybenzyl, and R⁶ and R⁷ combine to form a benzylidene or substituted benzylidene ring; X is O, S, or N; R¹ is alkyl, substituted alkyl, aryl, substituted aryl, PEG, substituted PEG, acyl or substituted acyl; and R² is levulinoyl, 4-chlorobenzoyl, benzoyl, 4-methylbenzoyl, acetyl or pivaloyl.

25. A compound according to claim 23, in which X is oxygen; R¹ is 3,4-methylenedioxybenzyl; R² is H, 4-chlorobenzoyl, pivaloyl, acetyl, levulinoyl, benzoyl or chloroacetyl; R³ and R⁴ either combine to become a benzylidene ring or are independently H, benzyl or substituted benzyl; and R⁵, R⁶ and R⁷ may be H, benzyl, 4-chlorobenzyl, 4-methoxybenzyl, 4-acetamidobenzyl, azidobenzyl or 3,4-methylenedioxybenzyl.

26. A compound according to claim 23, in which X is oxygen; R¹ is 2-[2-(2-thiobenzoyl)-ethoxy]ethyl or 2-[2-(2-thiobiphenylcabonyl)ethoxy]; R² is H, 4-chlorobenzoyl, pivaloyl, acetyl, levulinoyl, benzoyl or chloroacetyl; R³ and R⁴ combine to form a benzylidene ring, or are independently H, benzyl, 4-chlorobenzyl, 4-methoxybenzyl, 4-acetamidobenzyl, azidobenzyl or 3,4-methylenedioxybenzyl; R⁵ is H, benzyl, 4-chlorobenzyl, 4-methoxybenzyl, 4-acetamidobenzyl, azidobenzyl or 3,4-methylenedioxybenzyl; and R⁶ and R⁷ combine to become a benzylidene ring or are independently H, benzyl, 4-chlorobenzyl, 4-methoxybenzyl, 4-acetamidobenzyl, azidobenzyl or 3,4-methylenedioxybenzyl.

27. A compound according to claim 23, in which X is sulphur; R¹ is alkyl, substituted alkyl, aryl or substituted aryl; R³ and R⁴ combine to form a benzylidene ring; R⁵, R⁶ and R⁷ are benzyl; and R² is levulinoyl, 4-chlorobenzoyl, benzoyl, acetyl or pivaloyl,
with the proviso that when R¹ is phenyl, R² is not levulinoyl.

28. A compound according to claim 23, in which X is oxygen; R¹ is 2-[2-(2-thiobenzoyl)ethoxy]ethyl or 2-[2-(2-thiobiphenylcabonyl)ethoxy]; R² is H or 4-chlorobenzoyl; R³ and R⁴ are H or combine to form a benzylidene ring; R⁵ is H or 3,4-methylenedioxybenzyl; and R⁶ and R⁷ are both H, or combine to form a benzylidene ring.

29. A-compound according to claim 23, in which X is S, R¹ is methyl; R² is 4-chlorobenzoyl; R³ and R⁴ combine to form a benzylidene ring; and R⁵, R⁶ and R⁷ are each 4-chlorobenzyl.

30. A compound according to claim 23, in which X is oxygen; R¹ is 3,4-methylenedioxybenzyl; R² is 4-chlorobenzoyl or H; R³ and R⁴ combine to form a benzylidene ring or are both H; and R⁵, R⁶ and R⁷ are independently 4-chlorobenzyl or H.

31. A compound of general formula IX: 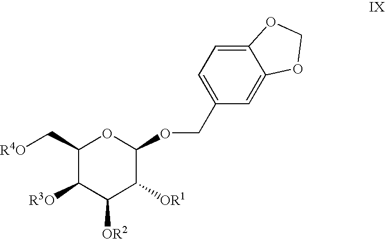
in which R¹ is 4-chlorobenzoyl, pivaloyl, acetyl, levulinoyl, benzoyl or chloroacetyl;
R² is H, benzyl, 4-chlorobenzyl, 4-methoxybenzyl, 4-acetamidobenzyl, azidobenzyl, 3,4-methylenedioxybenzyl, Fmoc, levulinoyl, acetyl or chloroacetyl; and
R³ and R⁴ combine to form a benzylidene ring, or are independently H, benzyl, 4-chlorobenzyl, 4-methoxybenzyl, 4-acetamidobenzyl, azidobenzyl or 3,4-methylenedioxybenzyl.

32. A compound according to claim 31, in which R¹ is 4-chlorobenzoyl, R² is H, and R³ and R⁴ combine to form a benzylidene ring.

33. A polyethyleneglycol(PEG)-linked disaccharide of General Formula X or a trisaccharide of General Formula XI: 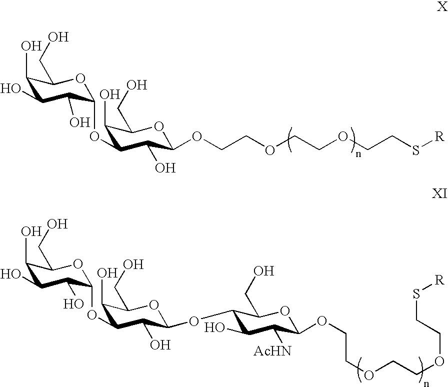
in which R is hydrogen or acyl, and n is an integer of from 1 to 3.

34. A compound of Formula XI according to claim which is 2-[2-(2-thiobiphenylcarbonyl)ethoxy]-ethyl 3-O-(α-D-galactopyranosyl)-α-galactopyranoside.

35. A compound of general formula XII: 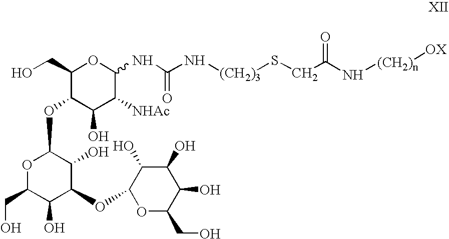
in which X is a solid support, and n is an integer of from 3 to 6.

36. A compound according to claim 35, in which X is Sepharose.

37. A compound according to claim 35, in which X is silica gel.

38. A method of synthesis of a disaccharide or trisaccharide, comprising the step of using a compound according to any one of claims 1 to 32 as an intermediate.

39. A method according to claim 38, in which the disaccharide or trisaccharide is selected from the group consisting of
(a) a compound of General Formula X, General Formula XI or General Formula XII;
(b) α-D-galactopyranosyl-(1→3)-β-D-galactopyranosyl-(1→4)-N-acetyl-D-glucosamine (Galα(1→43)Galβ(1→4)GlcNAc);
(c) α-D-galactopyranosyl-(1→3)-β-D-galactopyranose (Galα(1→43)Gal); and
(d) β-D-galactopyranosyl-(1→4)-N-acetyl-D-glucosamine (Galβ(1→4)GlcNAc).

40. A method according to claim 38 or claim 39, in which the compound is of General Formula X or XI, and the intermediate compound is of General Formula V.

41. A method according to claim 38, in which the compound is of General Formula VI, and the intermediate compound is of General Formula I.

42. A method of preventing or reducing a hyperacute rejection response associated with xenotransplantation, comprising the step of administering an effective dose of thioalkyl Galα-(1→3)Gal or thioalkyl Galα(1→3)Galβ(1→4)GlcNAc to a subject in need of such treatment.

43. A method of preventing or reducing hyperacute rejection associated with xenotransplantation, comprising the steps of
a) removing plasma from a patient who is to undergo xenotransplantation;
b) exposing the plasma to thioalkyl Galα(1→3)Gal or thioalkyl Galα(1→3)Galβ(1→4)GlcNAc linked to a solid support, and
c) reinfusing the thus-treated plasma into the patient.

44. A method of depleting anti-Galα(1→3)Gal antibodies from a plasma or serum sample, comprising the step of exposing the plasma or serum to thioalkyl Galα(1→3)Gal or thioalkyl Galα(1→3)Galβ(1→4)GlcNAc linked to a solid support.

45. A method of treatment of C. difficile infection, comprising the step of administering an effective amount of α-D-galactopyranosyl-(1→43)-β-D-galacto-pyranosyl-(1→4)-N-acetyl-D-glucosamine (Galα(1→3)Galβ(1→4)GlcNAc) or of thioalkyl Galα(1→3)Galβ(1→44)GlcNAc to a subject in need of such treatment.

46. A method according to claim 45, in which the Galα(1→3)Galβ(1β4)GlcNAc) or thioalkyl Galα(1→3)Galβ(1→4)GlcNAc, is linked to a solid support.

47. A method according to claim 45, in which the solid support is a multidentate ligand or a dendrimer compound.
